(19) United States
(12) Patent Application Publication
(10) Pub. No.: US 2017/0209576 A1
(43) Pub. Date: Jul. 27, 2017

(54) DELIVERY COMPOSITIONS AND METHODS

(71) Applicant: REGENTS OF THE UNIVERSITY OF MINNESOTA, MINNEAPOLIS, MN (US)

(72) Inventors: Joseph Anthony Zasadzinski, Chanhassen, MN (US); Natalie Anne Forbes, Crosby, TX (US)

(21) Appl. No.: 15/480,536

(22) Filed: Apr. 6, 2017

Related U.S. Application Data
(62) Division of application No. 14/674,353, filed on Mar. 31, 2015.
(60) Provisional application No. 61/972,825, filed on Mar. 31, 2014.

(51) Int. Cl. A61K 41/00; A61N 5/06; A61K 49/00; A61K 9/127; A61K 31/704

(52) U.S. Cl. CPC A61K 41/0028; A61K 9/1271; A61K 31/704; A61K 49/0043; A61N 5/062; A61N 2005/0659; A61N 2005/067

(57) ABSTRACT

This disclosure describes composition and methods for delivering a substance to a subject. Generally, the compositions include a liposome that includes a lysolipid and a cargo composition at least partially encapsulated by the liposome; and a reversibly heatable component coupled to the liposome. Generally, the method includes administering such a composition to a subject and causing localized release of the cargo composition by heating the reversibly heatable component of a localized portion of the composition.

### CROSS-REFERENCE TO RELATED APPLICATION

This application is a divisional application of U.S. patent application Ser. No. 14/674,353, filed Mar. 31, 2015, which claims priority to U.S. Provisional Patent Application Ser. No. 61/972,825, filed Mar. 31, 2014, each of which is incorporated herein by reference.

### GOVERNMENT FUNDING

This invention was made with government support under RO1 EB012637 awarded by the National Institutes of Health. The government has certain rights in the invention.

### SUMMARY

This disclosure describes, in one aspect, a composition that generally includes a liposome that includes a lysolipid and a cargo composition at least partially encapsulated by the liposome; and a reversibly heatable component that is coupled to the liposome. In some embodiments, the liposome further includes a PEG-lipid.

In some embodiments, the cargo composition includes a drug or a detectable signal.

In some embodiments, the reversibly heatable component is coupled to the liposome through a covalent bond. In some embodiments, the reversibly heatable component is coupled to the liposome by encapsulation in the liposome.

In some embodiments, the reversibly heatable component includes a nanoshell, a nanocube, a nanoparticle, a metal, and/or a copper sulfide nanoparticle.

In some embodiments, the reversibly heatable component is tuned to absorb near infrared radiation.

In some embodiments, the lysolipid is monopalmitoyl phosphatidyl choline (MPPC).

In another aspect, this disclosure describes a method for delivering a composition to a subject. Generally, the method includes administering to a subject any composition described above and causing localized release of the cargo composition by heating the reversibly heatable component of a localized portion of the composition.

In some embodiments, the method includes exposing a localized portion of the subject to near infrared radiation.

In some embodiments, the method includes stopping the release of the cargo composition by stopping the heating of the reversibly heatable component.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Schematic diagram of a lysolipid temperature sensitive liposome/hollow gold nanoshell (LTSL/HGN) hybrid nanocarrier. Typically, two or more hollow gold nanoshells are coupled via thiol-PEG-lipid linkers to the LTSL membrane. At 37° C., the LTSL membrane is in the solid phase, with low permeability to hydrophilic drugs. The hollow gold nanoshells adsorb near infrared (NIR) light and convert the light energy into heat, which raises the membrane temperature, initiating lysolipid-stabilized pores at membrane solid-fluid coexistence, which dramatically increase the membrane permeability. Release can be initiated, terminated or modified within seconds.

FIG. 2. Data characterizing liposomes. (A) Carboxyfluorescein (CF) release after 2.5 minutes at a given bulk temperature from dipalmitoylphosphatidylcholine (DPPC): 4 mol % 1,2-Distearoyl-sn-glycero-3-phosphoethanolamine (DSPE)-PEG²⁰⁰⁰ liposomes after modification with monopalmitoyl phosphatidyl choline (MPPC) and 1,2-Distearoyl-sn-glycero-3-phosphocholine (DSPC) as follows: 0 mol % MPPC and 0 mol % DSPC (squares); 15 mol % MPPC and 0 mol % DSPC (triangles), 15 mol % MPPC and 30 mol % DSPC (circles). MPPC induces a permeability transition near the solid-fluid coexistence temperature. DSPC raises the coexistence temperature of DPPC. (B) Cryo-TEM images of extruded liposomes formed from DPPC:4 mol % DSPE-PEG²⁰⁰⁰ on addition of MPPC. For 0 mol % and 10 mol % MPPC, liposomes are smooth with some faceting. For 15 mol % MPPC, the liposomes are more faceted than smooth. 20 mol % MPPC destabilizes the liposome structure by reducing the energy of the bilayer edge and stabilizing discs (shown by arrows). This mechanism confirms the release mechanism via MPPC stabilized pores shown in FIG. 1.

FIG. 3. Data characterizing nanoshells and LTSL/HGN. (A) Cryo-TEM images showing nanoshells tethered to the exterior membrane. (B) Cryo-TEM images showing nanoshells freely suspended in solution. (C) LTSL/HGN were irradiated for 2.5 minutes in a chamber controlled at 37° C. Release was observed at lower laser power when the hollow gold nanoshells were tethered to the membrane than when they were freely suspended in solution, and minimal release was observed in the absence of hollow gold nanoshells. The hollow gold nanoshells provide a collective heating effect, which raises the bulk solution temperature, as well as a local heating effect due to the temperature gradient surrounding the individual hollow gold nanoshell. This local heating effect causes the tethered hollow gold nanoshell to induce release from the lysolipid temperature sensitive liposome (LTSL) at lower power than the freely suspended gold nanoshells.

FIG. 4. Data characterizing nanoshells and LTSL/HGN. (A) Samples of different hollow gold nanoshell concentrations were held at 37° C., irradiated for 2.5 minutes with various laser powers, and the final bulk temperature was measured. (B) Carboxyfluorescein release profiles for LTSL/HGN initially held at the temperature given by the dotted line after irradiation with 1.2 W/cm² of laser power (filled squares) for 2.5 minutes plotted against the final bulk temperature compared to carboxyfluorescein release profiles for LTSL/HGN without laser irradiation (open squares). Irradiated LTSL/HGN release their contents at lower bulk temperatures than a non-irradiated sample, which gives an estimate of the effects of local heating on release (right arrow) relative to the collective effect of the hollow gold nanoshell on raising the sample temperature (left arrow). (C) Cumulative heating under irradiation showing the contributions from bulk and localized heating; the bulk heating is dependent on the hollow gold nanoshell concentration; the localized heating does not depend on hollow gold nanoshell concentration.

FIG. 5. Data characterizing LTSL/HGN. The rate and extent of release is controlled by laser power density. (A) The rate of release from LTSL/HGN depends on laser power density, with more rapid release at higher power density and more gradual release at lower power density. (B) Irradiation does not irreversibly damage liposomes. Irradiation for 30 seconds at 1.6 W/cm² after 22 hours at 37° C. showed a release of ˜50% of the LTSL/HGN contents. However, no additional release was observed for the following 24 hours, at which time the sample was re-irradiated for 60 seconds at 1.6 W/cm² laser power. A total of 90% of the LTSL/HGN contents were released after this second irradiation. This confirms that the release mechanism is by the production of transient pores, stabilized by the MPPC and DSPE-PEG²⁰⁰⁰ and not by an irreversible solubilization of the MPPC.

FIG. 6. Cryo-TEM images. (A,B) Cryo-TEM images of doxorubicin containing LTSL/HGN prior to irradiation and contents release. Multiple HGN are tethered to each LTSL. The arrows point to doxorubicin precipitates. Compare to FIGS. 3A and 3B. (C, D) After three minutes of 1.2 W/cm² laser irradiation with 800 nm NIR light, the LTSL remain sealed and there is no difference in the tethering of the HGN to the LTSL. The HGN retain their hollow shape and ˜40 nm diameter. Only the doxorubicin precipitates have disappeared, consistent with release during radiation. These images are consistent with only minor changes in temperature around the HGN during irradiation and a permeability transition due to transient pores opening up and closing with temperature.

FIG. 7. Data showing the effect of LTSL/HGN on cancer cell lines. (A) Cell death measured 48 hours after introduction of LTSL/HGN to PPC-1 prostate cancer cells at different liposomal doxorubicin concentrations (squares); and after three minutes of laser irradiation at 0.8 (triangle) and 1.2 W/cm² (diamond) laser intensity for 0.25 μM LTSL/HGN doxorubicin. More than 50 times the liposomal doxorubicin concentration is required to affect the same level of cell toxicity over 48 hours as the laser-released doxorubicin (p<0.05). This shows the excellent retention of doxorubicin in the LTSL/HGN, as well as demonstrating the difficulty reconciling good doxorubicin retention and therapeutic levels of drug release. (B) Cell death measured 5 hours, 24 hours, or 48 hours following irradiation. Cells were treated with 0.25 μM doxorubicin encapsulated within LTSL/HGN and irradiated for three minutes at 0.8 (triangles) or 1.2 W/cm² (diamonds); with 0.25 μM free doxorubicin without irradiation (circles) or with 0.25 μM doxorubicin in LTSL/HGN without irradiation (squares). The combination of irradiation and doxorubicin resulted in twice the cell killing than the equivalent free doxorubicin concentration. (C) Total cell killing after 48 hours for all samples and controls with and without irradiation. Irradiation of the LTSL/HGN/doxorubicin construct provided ˜90% cell killing compared to ˜50% cell killing for the same concentration of free doxorubicin (p<0.05). LTSL without hollow gold nanoshell showed minor cell toxicity due to slow release from the liposomes without bulk heating. Bulk heating due to irradiating hollow gold nanoshell alone showed only minor cell toxicity. Irradiation with NIR light with no hollow gold nanoshell showed minimal toxicity as did blank liposomes.

FIG. 8: Transmission electron microscope image and size distribution of monodisperse silver template particles prepared by the polyol process from silver nitrate precursor.

FIG. 9. TEM image and size distribution of hollow gold nanoshells prepared by galvanic replacement of gold for silver using the spherical silver template particles prepared from silver nitrate precursor.

FIG. 10. Data characterizing constructs. (A) Silver nanocube templates made by polyol method with silver trifluoroacetate as precursor. (B,C) Hollow gold nanocages (HGN) of 36±4 nm and 15.8±5 nm size made by galvanic replacement of gold on the silver templates in (A); the insets show the enlarged structure of the cubes. (D) Absorption spectra of HGN. The maximum absorption can be shifted to lower wavelengths by increasing the gold thickness to edge length ratio.

FIG. 11. Copper sulfide (CuS) nanoparticle absorbance and TEM images. Absorption peaks around 950 nm, and TEM images show small, solid spherical particles with <10 nm diameter. CuS absorption does not depend on the shape of the particles so they can be made smaller than the hollow gold nanoshells or nanocubes.

FIG. 12. Phase transitions in saturated DPPC or DPPG monolayers induced by ethanol. Below the gel-liquid crystalline temperature, Tₘ (41° C. for DPPC), the bilayer is crystalline and the hydrocarbon chains are tilted, forming the L_{β′} phase. The addition of ethanol swells the headgroup area until the chain tilt is eliminated in favor of maintaining tight packing, resulting in lipid chain interdigitation and the L_{βI} phase. The bilayers in the L_{βI} phase are much stiffer, causing the vesicles to break open and form extended, open bilayer sheets. The bilayers remain as planar sheets following the exchange of the 3M ethanol for water, but revert to the tilted L_{β′} phase. Heating above Tₘ results in chain expansion, leading to the fluid bilayer phase. The fluid bilayers quickly close on the surrounding solution, encapsulating nanoparticles or vesicles in the solution. (Kisak et al., Current Med. Chem. 2004, 11:199-219; Kisak et al., Langmuir, 2002, 18:284-8.) CuS or small hollow gold nanocubes can be trapped inside the vesicle membrane in this way.

FIG. 13. Interdigitation-Fusion Vesicle (IFV) synthesis process. Pure DPPC liposomes are converted to interdigitated sheets and then washed of excess ethanol (as in FIG. 12). The planar bilayer sheets are hydrated with buffer containing nanoparticles (either hollow gold nanoshells or nanocubes, or CuS nanoparticles) and small molecule drug or drug excipient (doxorubicin in this example) to be encapsulated. Micellar lysolipid can be added during hydration or to the pre-formed IFVs following hydration. Unencapsulated nanoparticles and small molecules are removed, typically by centrifugation, and the IFVs are then PEGylated with micellar DSPE-PEG²⁰⁰⁰ to create a thermosensitive liposome with internalized heatable elements.

FIG. 14. Mechanism of lysolipid transfer into pre-formed liposomes from micellar lysolipid. Lysolipid monomers rapidly partition into the outer leaflet of the liposome bilayer. Lysolipid micelles provide a reservoir to maintain monomeric lysolipid at the critical micelle concentration (CMC). Lysolipid is exchanged from the outer to the inner bilayer leaflet through lipid flip-flop or transient defect structures.

FIG. 15. Sample heating by CuS nanoparticles encapsulated in Interdigitation-Fusion Vesicles (IFV) under irradiation with 800 nm NIR light at 7 W/cm² intensity. Sample temperature after five minutes is 40° C. for samples containing CuS nanoparticles, which is 2° C. higher than buffer irradiated under the same conditions.

FIG. 16. Release from the lysolipid-containing IFVs under irradiation. Dye release was observed only from lysolipid-containing IFVs with encapsulated CuS nanoparticles. DPPC IFV with non-encapsulated CuS nanoparticles did not release their contents despite comparable levels of heating. Without encapsulated CuS nanoparticles, lysolipid-containing IFVs did not reach their main transition temperature and therefore did not release the encapsulated dye.

FIG. 17. Data characterizing liposomes. (A) Cumulative carboxyfluorescein (CF) dye release in 2.5 minutes as a function of MPPC lysolipid mole fraction in DPPC liposomes. Without MPPC, the permeability does not change with temperature. With MPPC, the permeability undergoes a step-change at 39-40° C. (B) CF dye release as a function of time at a given bulk sample temperature for 15:85 mol % MPPC: DPPC liposomes. At 37° C., there is negligible release, with complete release occurring in <5 minutes at 40° C.

FIG. 18. Data characterizing lysolipid partitioning and membrane permeability. (A) Lysolipid partitioning into DPPC liposomes as a function of total lysolipid concentration. Lysolipid (MPPC with 10 mol % NBD-lysolipid) is added to the DPPC liposome; Lyso X^{Total Lipids} is the lysolipid fraction of the total lipid concentration (lyso+DPPC) in solution. Lyso X^{Bilayer} is the mole fraction of MPPC in the bilayer. Partitioning is higher in the fluid phase relative to the gel phase of DPPC. (B) Membrane permeability determined by carboxyfluorescein dye release from the lysolipid-containing liposomes at 40° C. after 2.5 minutes. Membrane permeability only depends on the lysolipid mole fraction in the bilayer and not on how the lysolipid-liposomes were prepared. Thin film refers to mixing the lysolipid and DPPC before extruding the liposomes as in FIG. 17.

FIG. 19. Data characterizing lipid bilayers. (A) Micellar lysolipid was added to PEGylated liposomes at 10 mol % of the total lipid concentration. 100% surface coverage for methoxy-terminated DSPE-PEG²⁰⁰⁰ (mPEG 2000) was 5 mol % and 17 mol % for methoxy-terminated DSPE-PEG⁷⁵⁰ (mPEG 750). Lyoslipid partitioning into the liposome membranes decreased at 50% surface coverage to zero at 100% coverage. (B) The maximum concentration of lysolipid needed to destabilize liposomes decreased with PEGylation. 50% surface coverage of mPEG 750) has minor effects, but 50% surface coverage of mPEG 2000 reduces the allowable lysolipid mole fraction by half (C) Cryo-TEM images of DPPC with 4 mol % DSPE-PEG²⁰⁰⁰ with increasing lysolipid mole fraction. Lysolipid plus DSPE-PEG²⁰⁰⁰ leads to faceted liposomes that break up into discs (arrows).

FIG. 20. Data characterizing liposomes. (A) The zeta potential of DPPC liposomes becomes more negative as the fraction of negatively-charged DPPG (inset) increases. However, partitioning of MPPC lysolipid into the membrane is independent of the zeta potential. (B) Deprotonation of the terminal amine group (inset) of DPPE at pH 9 leads to a decrease in zeta potential for 5:95 DPPE:DPPC liposomes. However, the surface charge did not impact MPPC lysolipid partitioning into the membrane.

FIG. 21. Transfer of DSPE-PEG²⁰⁰⁰ into the liposomes at either 37° C. (gel phase, open circles) or 55° C. (fluid phase, filled squares) during 1 hour of incubation with solutions containing various DSPE-PEG²⁰⁰⁰ mol % relative to the total lipids. The maximum amount of DSPE-PEG²⁰⁰⁰ incorporation after 1 hr is about 5 mol %, which corresponds to the fully covered “mushroom” state.

### DETAILED DESCRIPTION OF ILLUSTRATIVE EMBODIMENTS

This disclosure describes a novel drug carrier that includes plasmonic hollow gold nanoshells (HGN) chemically tethered to a temperature-sensitive liposome. This disclosure also describes a novel drug carrier that includes CuS nanoparticles incorporated in a temperature-sensitive liposome. Continuous-wave irradiation by physiologically acceptable near infrared light (e.g., at 800 nm) at laser intensities an order of magnitude below that known to damage skin generates heating localized to the liposome membrane. The heating transiently increases the liposome permeability in an irradiation dose-dependent but reversible manner, resulting in rapid release of small molecule contents of the liposome. This enables precise control of contents release with low temperature gradients confined to areas irradiated by the laser focus. The combined effects of rapid local release and localized hyperthermia can provide a synergistic effect.

In the description that follows, the term “and/or” means one or all of the listed elements or a combination of any two or more of the listed elements; the terms “comprises” and variations thereof do not have a limiting meaning where these terms appear in the description and claims; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

One challenge still facing liposomal drug delivery is controlling drug release. Few methods are available to controllably switch from drug retention to varied levels of drug release, or switch from fast release back to drug retention. Optimizing both retention and release is difficult within the constraints of a single bilayer. (Kisak et al., Current Med. Chem., 2004, 11:199-219; Charrois et al., Biochim. Biophys. Acta., 2004, 1663:167-77; Zhu et al. Integr. Biol. 2013, 5:96-107; Boyer et al., ACS Nano, 2007, 1:176-82; Walker et al., Nature, 1997, 387:61-4; Wong et al., Adv. Materials, 2011, 23:2320-5.) For example, encapsulating doxorubicin within PEGylated liposomes can significantly reduce cardiotoxicity and can increase the half-life of doxorubicin in the systemic circulation due, at least in part, to optimized doxorubicin retention in the liposome. Efficacy did not improve as expected from the increased liposomal doxorubicin accumulation at the tumor site (O'Brien et al., Ann. Oncol., 2004, 15:440-449), however, at least in part because of slow drug release. Cisplatin incorporated into PEGylated liposomes can extend systemic half-life to 40-55 hours in humans, in comparison to the 15-20 minute clearance of free cisplatin. The formulation failed in human trials, however, at least in part because cisplatin was not released at a therapeutic rate from the liposomes, even though 5-fold to 10-fold greater liposome-encapsulated cisplatin accumulated in the tumors. This highlights the conflict between “retain” and “release”: factors that enhance drug retention usually work against an optimal rate of drug release.

“Thermosensitive liposomes” (TSL) and other stimuli-sensitive liposomes were developed in response to this conflict. Thermosensitive liposomes typically have a membrane composition designed to induce a step-change in permeability in response to modest changes in temperature above, for example, 37° C. One subset of thermosensitive liposomes are lysolipid thermosensitive liposomes, which typically contain dipalmitoylphosphatidylcholine (DPPC), a lysolipid (single-chained phospholipid), and lipid-conjugated PEG²⁰⁰⁰ (DSPE-PEG²⁰⁰⁰).

Lysolipid-TSL formulations (LTSL) have shown improved efficacy over other thermosensitive liposomes in a number of cancer cell lines in vitro and in vivo. Clinical trials are ongoing for treatment of liver and breast cancers with a combination of lysolipid thermosensitive liposomes and regional hyperthermia. Regional hyperthermia typically involves bulk heating with water baths, radiofrequency (RF), or microwave (MW) radiation, etc. (Needham et al., Cancer Res., 2000, 60:1197-201; Needham et al., Adv. Drug Delivery Rev., 2001, 53:285-305; Dewhirst et al., Surg. Oncol. Clin. N. Am., 2013, 22:545-61; Stauffer, Int. J. Hyperthermia, 2004, 20:671-677), often requiring invasive implants. Using regional hyperthermia to control the temperature profile and extent of heating can be difficult, especially in highly perfused tissues.

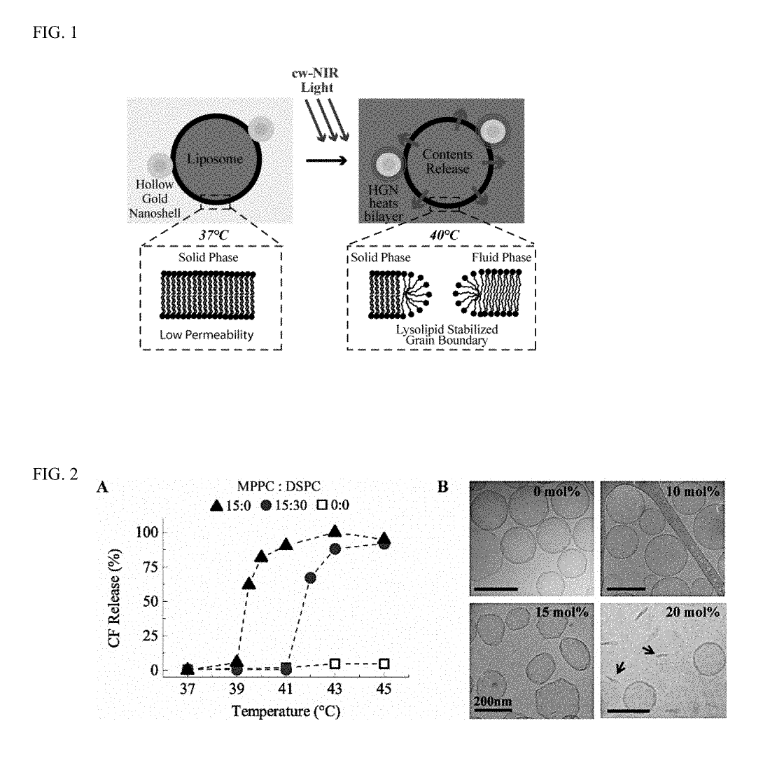

In contrast, this disclosure describes a non-invasive method to initiate, control, and even stop drug release via truly localized temperature control. PEG-stabilized plasmonic hollow gold nanoshells (HGN) (Prevo et al. Small, 2008, 4:1183-95; Wu et al., Methods in Enzymology, 2009, 464:279-307) are tethered to lysolipid thermosensitive liposomes via thiol-PEG-lipids to create a hybrid nanocarrier (FIG. 1). The LTSL/HGN are irradiated with continuous wave near infrared (NIR) laser light (e.g., at 800 nm) at intensities an order of magnitude less than that known to damage skin. The lower irradiation intensity is nevertheless sufficient to increase temperatures near the liposome membrane to rapidly release the liposome contents (FIG. 1).

One advantage of using NIR light to induce release the contents of the LTSL/HGN is that tissue, blood, etc. are relatively transparent to 650-950 nm wavelength light, allowing NIR transmission in soft tissues at depths up to 10 cm. (Agrawal et al., ACS Nano, 2011, 5:4919-26; Weissleder, Nature Biotechnology, 2001, 19:316-7.) Laser heating can be extremely localized and can induce a near instantaneous response, allowing the entire liposome contents to be released in seconds with minimal background heating. In contrast, regional hyperthermia, by definition, involves a significant degree of background heating. The liposome temperature can revert to ambient equally quickly when NIR irradiation stops, allowing the liposomes to re-seal, thereby making the drug release controllable—i.e., drug release can be stopped and re-started. Previous technologies rely upon hyperthermia to rupture liposome to release liposome contents and, therefore, cannot be stopped and restarted. Only the HGN/LTSL complexes that are directly irradiated by the irradiation are heated sufficiently to induce drug release, which further providing a precise targeting mechanism for drug delivery.

Rapid, controlled, NIR-induced doxorubicin release from HGN/LTSL results in irradiation-dose dependent killing of PPC-1 androgen resistant prostate cancer cells in vitro. Drug release is accomplished with three minutes of irradiation. Surprisingly, even this short irradiation time provides a synergistic heating and concentration effect that provides roughly double the killing efficiency of free doxorubicin at the same concentration. In contrast, passive permeation of doxorubicin from the liposomes required nearly two orders of magnitude higher total doxorubicin concentration to effect similar toxicity.

First, we used the self-quenching fluorescent dye, carboxyfluorescein (CF) as a model water-soluble drug for hollow-gold-nanoshell-driven contents release. Our goal was to provide 48 hours or more of carboxyfluorescein retention at 37° C. as well as total contents release within minutes at a temperature increase of 3-4° C. Continuous wave NIR light intensities of up to 12 W/cm² for five minutes do not damage skin (Ramadan et al., Small, 2012, 8:3143-50; Timko et al., Adv. Materials, 2010, 22:4925-43; Zhou et al., J. Am. Chem. Soc., 2010, 132:15351-8) which gives an upper limit for the light intensity that we chose to use to induce the required temperature change. Lysolipid thermosensitive liposomes were formed by self-assembly from mixtures of monopalmitoylphosphatidylcholine (MPPC), which matches the 16 carbon saturated alkane chain length of the double-tailed dipalmitoylphosphatidylcholine (DPPC), which has a gel to liquid crystal transition temperature of 41° C. All liposomes incorporated 4 mol % DSPE-PEG²⁰⁰⁰ for steric stabilization.

FIG. 2A shows that the fractional carboxyfluorescein release over 2.5 minutes is strongly dependent on the bulk sample temperature and can be readily modified by changes in liposome membrane composition. For DPPC liposomes with 4 mol % DSPE-PEG²⁰⁰⁰, there is minimal change in the rate of carboxyfluorescein release at the main phase transition temperature of 41° C.; the fluid L_{α} phase bilayer is only marginally more permeable than the ordered L_{β′} bilayer (Landon et al., Open Nanomedicine 1, 2011, 3:38-64) and there is no anomalous carboxyfluorescein release at the transition temperature (Li et al. J. Controlled Release, 2013, 168:142-50). Adding 15 mol % MPPC dramatically increased carboxyfluorescein release rates for temperatures≧39° C.; release was undetectable at 37° C. Further increases in temperature to 41° C. increased liposome permeability sufficiently so that 100% of the encapsulated carboxyfluorescein was released within 2.5 minutes (FIG. 2A).

The permeability transition can be shifted to higher temperature by adding DSPC to the liposome membrane. DPPC, which has two 16-carbon alkane chains, has a gel (L_{β′}) to liquid crystal (L_{α}) temperature of 41° C., while DSPC, with two 18-carbon alkane chains has a transition temperature of 54° C. DPPC and DSPC are completely miscible in both the fluid and gel states, so liposomes with intermediate transition temperatures can be obtained by altering the ratio of DSPC to DPPC. (Landon et al., Open Nanomedicine 1, 2011, 3:38-64; Ipsen et al. Biochim. Biophys. Acta, 1988, 944:121-34.) The inclusion of 30 mol % DSPC and 15 mol % MPPC in DPPC liposomes increased the temperature at which dye release was initiated by 2-3° C., from 39-40° C. to 41-42° C. (FIG. 1A), which is also the increase in temperature of the gel to liquid crystal transition for the 30% DSPC/DPPC mixture. As the phase transition is shifted towards higher temperatures, the liposomes become increasingly stable against leakage at 37° C. The release profile from the DSPC-containing liposomes with 15 mol % MPPC in the bilayer was nearly identical to those without DSPC, only shifted by 2-3° C.

The maximum lysolipid concentration may be limited by liposome stability (FIG. 2B), which also suggests the mechanisms behind the increased permeability at the phase transition. Cryo-TEM images of samples quenched from room temperature show that DPPC liposomes with 4 mol % DSPE-PEG²⁰⁰⁰ and 0 mol % MPPC have the typical, spheroidal shape of gel phase liposomes. The bilayer membranes are rough, likely due to faceting of the rigid, gel-phase membranes. Adding 10 mol % MPPC does not alter the liposome appearance significantly. However, 15 mol % MPPC enhances the faceting of the liposome membrane. The grain structure of the liposomes is more pronounced at 15 mol %, and the facets are more extensively flattened with sharper edges between the facets. This suggests that the MPPC is segregated to the high curvature regions at the grain boundaries. Further increasing MPPC to 20 mol % destabilized the liposomes and led to coexistence between bilayer discs (arrows) and intact liposomes.

The discs may be stabilized by the segregation of the MPPC and DSPE-PEG²⁰⁰⁰ to the disc perimeters, which lowers the energy associated with exposing the hydrophobic bilayer edge to water. (Jiang et al., Biophys. J., 2010, 98:2895-903; Jung et al., PNAS, 2002, 99:15318-22; Fromherz, Chem. Phys. Letters, 1983, 94:259-66.) Components like MPPC and DPSE-PEG²⁰⁰⁰, that prefer high-curvature, micellar structures on their own, can act as “edge-actants,” and may lower the energy associated with the bilayer edge. This edge energy may be less than the curvature energy associated with bending a gel-phase bilayer to form a closed vesicle, thereby stabilizing discs. Mixtures of DPPC with diheptanoylphosphatidylcholine, the seven carbon-long alkane version of DPPC, also form discs with the shorter-tail lipids stabilizing the edges. Molecular simulations show that stable and unstable bilayer pores can form spontaneously in bilayers with edge-actant concentrations less than that needed to break up the liposomes into discs. Such transient pores are likely the route through which small molecules are released. Such a mechanism would also allow for the pores to be re-sealed by lowering the temperature.

Local vs. Collective Heating

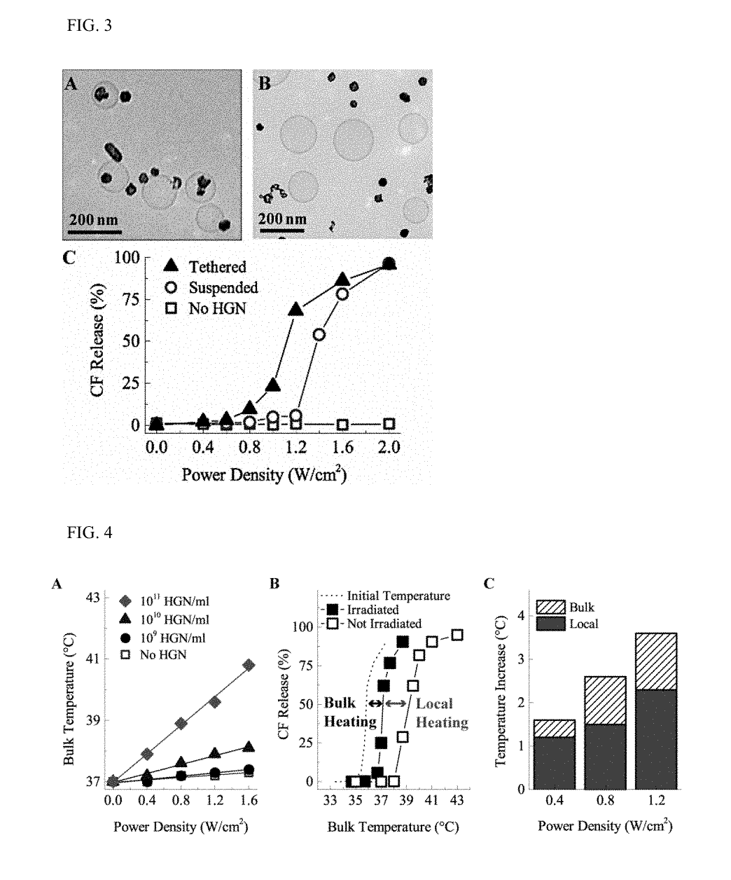

Instead of relying on regional hyperthermia to create the necessary bulk temperature increase to increase lysolipid thermosensitive liposome permeability as in FIG. 2A, we provide each lysolipid thermosensitive liposome with its own source of heat in the form of hollow gold nanoshells (HGN) physically tethered to the exterior of the liposome membrane. Each hollow gold nanoshell generates heat through the inter-conversion of the absorbed light energy from an 800 nm continuous wave laser into heat (FIG. 3). A small fraction of PEG-lipids on the lysolipid thermosensitive liposome bilayer exterior were modified with a sulfhydryl group that binds to the hollow gold nanoshells. Simple mixing of the lysolipid thermosensitive liposomes with hollow gold nanoshell in saline led to the tethered HGN/LTSL (FIG. 3A). The physical attachment of the nanoshells to the liposome was confirmed by cryo-TEM images (FIG. 3A).

Tethering significantly reduces the laser power needed to induce carboxyfluorescein release from lysolipid thermosensitive liposomes compared to free hollow gold nanoshells (FIG. 3C). For the tethered LTSL/HGN, dye release begins at 0.8 W/cm² laser power density, while >1.2 W/cm² was required to initiate dye release from the lysolipid thermosensitive liposomes with the free hollow gold nanoshells. For both cases, however, this intensity is an order of magnitude less than the 12 W/cm² light intensity known to damage skin. (Ramadan et al., Small, 2012, 8:3143-50; Timko et al., Adv. Materials, 2010, 22:4925-43; Zhou et al., J. Am. Chem. Soc., 2010, 132:15351-8.) Dye release for the untethered nanoshells was due primarily to an increase in the bulk solution temperature. No dye release was observed over this time scale in lysolipid thermosensitive liposomes without nanoshells and NIR irradiation. The steady state sample temperature reached after irradiation for extended times (>10 minutes) was the same for the tethered and untethered samples, which confirms that the total hollow gold nanoshell concentrations were the same in both samples (FIG. 4A).

The rate of heat generation by the hollow gold nanoshells is proportional to both the laser intensity and the hollow gold nanoshell concentration (FIG. 4A). Hollow gold nanoshells at various concentrations suspended in saline in a glass cuvette were placed within a temperature controlled chamber at 37° C. and irradiated with increasing laser intensities for 2.5 minutes. Saline without hollow gold nanoshells did not increase in temperature significantly at any level of laser irradiation. With hollow gold nanoshells in the solution, however, the bulk solution temperature increased linearly with laser intensity (FIG. 4A). This heating is the cumulative effect of the conversion of the light energy of the laser into heat, which diffuses throughout the sample. It should not be confused with the temperature of the hollow gold nanoshell itself or the temperature profile in the immediate vicinity of the hollow gold nanoshell during irradiation.(Baffou et al., Phys. Rev. B., 2011. 84:035415; Baffou et al. ACS Nano., 2010, 4:709-16.) The bulk sample temperature increases with nanoshell concentration over the range of 10⁹-10¹¹ HGN/ml. At the maximum light intensity of 1.6 W/cm², negligible bulk heating was observed for a sample with 10⁹ HGN/ml—about 1° C. for 10¹⁰ HGN/m1)—while for 10¹¹ HGN/ml there was a 4° C. increase in temperature after 2.5 minutes of light exposure.

The nanoshell concentration for the LTSL/HGN samples shown in FIG. 3C and FIG. 4B is approximately 10¹⁰ HGN/ml, which FIG. 4A shows leads to an increase in bulk sample temperature of less than 1° C. in 2.5 minutes for all the laser powers tested. For 10¹⁰ HGN/ml, the slope of the heating curve (FIG. 4A) gives an effective bulk heating rate of approximately 0.7° C./(W/cm²). Extrapolating this result suggests that a laser intensity of approximately 4 W/cm² can achieve the 3° C. increase in bulk temperature within 2.5 minutes necessary to trigger release of the contents of lysolipid thermosensitive liposomes.

FIG. 3C shows that contents release is triggered at much lower laser intensities for the lysolipid thermosensitive liposomes with tethered hollow gold nanoshells, however, showing that the temperature of the lysolipid thermosensitive liposome membrane in the vicinity of the hollow gold nanoshell is greater than the bulk sample temperature during irradiation. The “hot” hollow gold nanoshells tethered to the liposome are less than 5 nm from the liposome bilayer, and cause a localized greater increase in membrane temperature relative to the bulk sample temperature. This localized heating of the tethered hollow gold nanoshell is independent of the hollow gold nanoshell concentration—each lysolipid thermosensitive liposome is heated above the bulk temperature by its own tethered hot hollow gold nanoshell.

To differentiate between the localized and collective heating effects of the nanoshells, FIG. 4B shows the dye release from irradiated LTSL/HGN compared to non-irradiated LTSL/HGN. Both samples were held in a temperature-controlled chamber. For the non-irradiated LTSL/HGN, an external heat source was applied to rapidly increase the bulk temperature of the sample to a particular value, during which time the carboxyfluorescein fluorescence was measured. The carboxyfluorescein fluorescence was then determined after 2.5 minutes at the desired temperature (open squares). The non-irradiated sample has a release profile similar to that of 15 mol % MPPC sample shown in FIG. 2A; the chemical tethering of the hollow gold nanoshell to the lysolipid thermosensitive liposome did not alter the carboxyfluorescein release at a given temperature.

To estimate the local heating effect, we assumed that at a given carboxyfluorescein release rate, the membrane temperatures would also be the same. For the irradiated sample, the bulk sample temperature was controlled at the beginning of irradiation (dotted lines in FIG. 4B). The samples were exposed to 800 nm NIR light of varied intensity (1.2 W/cm² for the data in FIG. 4B; other laser intensities not shown), and the sample temperature and fractional carboxyfluorescein release were determined after 2.5 minutes of irradiation (closed squares). The change in temperature in the irradiated sample was due to the collective effect of the inter-conversion of light to heat via the irradiated hollow gold nanoshells. However, as can be seen in FIG. 4B, carboxyfluorescein is released from the LTSL/HGN at a lower bulk temperature than the non-irradiated LTSL/HGN. The difference in temperature between the two carboxyfluorescein release curves at a given carboxyfluorescein release rate is due to the localized membrane heating (right arrow)—i.e., there is a difference between the lysolipid thermosensitive liposome membrane temperature and the bulk temperature due to the proximity to the irradiated hollow gold nanoshell. Over the entire range, the irradiated sample shows equivalent contents release at 2-3° C. lower bulk sample temperatures. This is consistent with theoretical calculations of the steady state temperature distribution around laser-heated nanoparticles.

FIG. 4B shows the relative contributions to the effective membrane temperature from the local and collective effects of irradiation on the LTSL/HGN for the sample irradiated with 1.2 W/cm² at 50% dye release (FIG. 4B, arrows). The left arrow shows the collective effect of the hollow gold nanoshell heating the entire sample, while the right arrow shows the local temperature difference between the lysolipid thermosensitive liposome membrane and the bulk solution. FIG. 4C shows that at 1.2 W/cm², the lysolipid thermosensitive liposome membrane experiences an effective temperature increase of 3.6° C. above the initial temperature of 35.7° C., which leads to 50% release of the encapsulated dye within 2.5 minutes. This is consistent with FIG. 2A that shows the effective lysolipid thermosensitive liposome temperature (15 mol % MPPC) must be at least 39° C. to transition to the highly permeable state. At 0.4 W/cm² irradiation intensity, the lysolipid thermosensitive liposome membrane experiences an effective temperature increase of less than 1.5° C., resulting in a lysolipid thermosensitive liposome temperature of less than 39° C. and minimal release was observed, again consistent with FIG. 2A. At 0.8 W/cm², the liposome membrane with tethered hollow gold nanoshell experiences an effective temperature increase of 2.5° C., which is sufficient to initiate dye release.

The localized heating is the origin of the differing power intensities required to release carboxyfluorescein for the tethered and untethered hollow gold nanoshells. For a laser intensity of 1.2 W/cm², the tethered hollow gold nanoshell released approximately 70% of the carboxyfluorescein in 2.5 minutes. In contrast, the sample with untethered hollow gold nanoshell showed minimal release. The untethered hollow gold nanoshells contribute to raising the bulk temperature, and less to changing the local lysolipid thermosensitive liposome membrane temperatures. Both the tethered and untethered samples have the same hollow gold nanoshell concentration and thus experience the same bulk temperature increase of approximately 1.3° C. in 2.5 minutes. However, the bulk temperature increase is below the threshold membrane temperature (37° C.+1.3° C.) required for lysolipid thermosensitive liposome contents release from FIG. 2A; localized heating is also required. Thus, physically tethering the nanoshells to the liposome surface ensures that they will initiate release at much lower light intensity, as well as localizing to the same site as the lysolipid thermosensitive liposome. This leads to more efficient contents release under irradiation, regardless of the net concentration of hollow gold nanoshell in the irradiated volume.

The threshold laser power density is the lowest laser intensity that will ensure that the localized heating of the hollow gold nanoshells (FIG. 4C) is sufficient to raise the liposome membrane temperature to greater than 39° C. This will enable contents release regardless of the concentration at which the LTST/HGN have accumulated at the treatment site. For the 81:15:4 DPPC:IVIPPC:DSPE-PEG²⁰⁰⁰ liposomes, this laser threshold power density is 1.2 W/cm². At this laser intensity, the localized nanoshell heating raises the liposome membrane temperature to 2.3° C. above that of the surrounding sample temperature, which is sufficient to increase membrane permeability and trigger release. This power intensity is an order of magnitude below the 12 W/cm² threshold observed in previous studies that caused skin irritation in animals. (Ramadan et al., Small, 2012, 8:3143-50; Timko et al., Adv. Materials, 2010, 22:4925-43; Zhou et al., J. Am. Chem. Soc., 2010, 132:15351-8.)

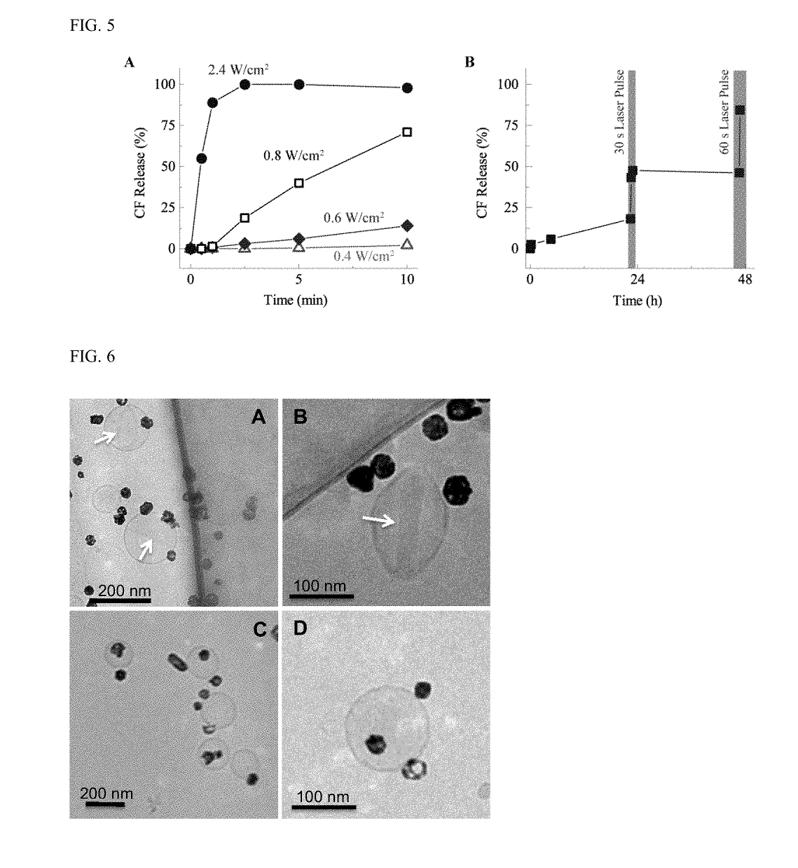

Localized heating also means a rapid temperature response on both starting and stopping irradiation. The steady state temperature distribution around a hollow gold nanoshell of radius R on irradiation is established after tₛₛ˜10R²/α_{w}, in which α_{w}=0.14×10⁻⁶ m²/s is the thermal diffusivity of water. For a 40 nm diameter hollow gold nanoshell, τₛₛ<0.1 μs. For 2.4 W/cm² light intensity, dye release begins almost instantly after irradiation starts and is 90% complete within 30 seconds (FIG. 5A). Without the effects of localized heating, the entire sample volume would need to increase in temperature to greater than 39° C. to reach the permeability transition before any carboxyfluorescein release would begin. Extrapolating from FIG. 4A for a hollow gold nanoshell concentration of 10¹⁰ HGN/ml, it would take approximately five minutes to reach 39° C. FIG. 5A also shows that the release rate is proportional to the laser intensity. Release first occurs at a laser power density of 0.6 W/cm². At this light intensity, approximately 15% of the total carboxyfluorescein was released over 10 minutes, compared to the 90% release in 30 second at 2.4 W/cm². The release rate can be tailored by controlling the light intensity, while the total release can be controlled by the duration of irradiation.

The rapid response of the hollow gold nanoshell to irradiation also means that the local heating dissipates rapidly after irradiation stops. FIG. 5B shows that staged release of LTSL/HGN contents is possible. LTSL/HGN encapsulating carboxyfluorescein were held at 37° C. for 22 hours, followed by 30 seconds of irradiation at an intensity of 1.6 W/cm². Prior to irradiation, the LTSL/HGN effectively retained the encapsulated dye, approximately 10% of the dye was lost due to passive leakage, which is similar to values in the literature for other lysolipid thermosensitive liposomes. On irradiation, 35% of the encapsulated dye was released during a 30-second irradiation. The LTSL/HGN were not irreversibly altered by the irradiation or the carboxyfluorescein release; the remaining carboxyfluorescein was retained within the LTSL/HGN for the following 24 hours.

In addition to carboxyfluorescein release starting immediately on irradiation, FIG. 5B shows that dye release stops equally quickly when irradiation stopped. This is consistent with the release mechanism being the formation of transient but reversible pores near the phase transition temperature (FIG. 1) initiated by the local heating effects of the hollow gold nanoshells. Irradiation does not disrupt lysolipid thermosensitive liposome integrity; the lysolipid thermosensitive liposomes are just as impermeable to carboxyfluorescein release after the first irradiation as before irradiation, as indicated by the retention of the remaining encapsulated dye for the 24 hours between the first and second irradiation. Suggestions in the literature that the lysolipid is ejected from the lysolipid thermosensitive liposome during release (Landon et al. Open Nanomedicine J., 2011, 3:38-64) are likely incorrect as the remaining dye was released by a second 60 second pulse of 1.6 W/cm² intensity after 46 hours at 37° C. Had the lysolipid been ejected from the lysolipid thermosensitive liposome, drug release may have stopped after the first irradiation, but would not have restarted at the same rate on the second irradiation.

FIG. 6 shows cryo-TEM images taken following irradiation that show the liposomes remain as closed spherical structures and the nanoshells retained their structure of a thin shell with a hollow core. Irradiation with the same average light intensity from femtosecond pulsed lasers establish large temperature gradients, rupturing the liposomes via formation of nanobubbles, while annealing the hollow nanoshells into solid gold nanoparticles. The hollow gold nanoshells still strongly absorb near infrared light after the carboxyfluorescein is released, which means that subsequent irradiation can be used to induce a bulk temperature increase.

### Doxorubicin Release in an In Vitro Prostate Cancer Cell Model

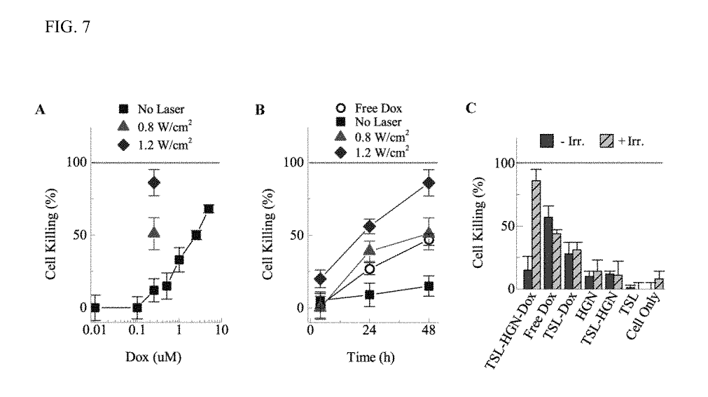

To demonstrate the benefits of light-triggered release, doxorubicin was encapsulated within the LTSL/HGN using pH gradient loading with ammonium sulfate. (Landon et al. Open Nanomedicine J., 2011, 3:38-64.) Doxorubicin release from lysolipid thermosensitive liposomes has been shown using multiple forms of bulk hyperthermia and has shown synergistic effects of hyperthermia and doxorubicin in a variety of cancer cell lines. FIG. 7A shows that without irradiation, doxorubicin gradually leaks from the LTSL/HGN over 48 hours at 37° C. in cultures of androgen-resistant PPC-1 prostate cancer cells (squares); there was minimal toxicity below 0.25 μM doxorubicin concentration within the LTSL/HGN. 10 μM doxorubicin in LTSL/HGN was required to induce significant toxicity without irradiation, showing the excellent retention of the doxorubicin in the LTSL/HGN. The data also shows the essential contradiction between drug retention and therapeutic levels of drug release that limits conventional liposome drug delivery. The better doxorubicin is retained in the liposome, the harder it is to achieve a toxic level of active doxorubicin, even when the doxorubicin is allowed to accumulate within the confines of the small volume of the cell culture well. It is likely that this difficulty would be exacerbated in vivo, as it is difficult to confine the doxorubicin once it is released. (Manzour et al., Cancer Res., 2012, 72:5566-75.)

Three minutes of irradiation of LTSL/HGN with 0.25 μM total doxorubicin with 0.8 (triangle) or 1.2 W/cm² (diamond) 800 nm light showed a significant, light-dose dependent increase in toxicity compared to the non-irradiated LTSL/HGN, even at 10 μM encapsulated doxorubicin. At 0.8 W/cm², 55±5% of the PPC-1 cells were killed; 86±5% of the PPC-1 cells were killed when irradiated at 1.2 W/cm². This is compared to the 10±5% killing by non-irradiated LTSL/HGN doxorubicin and the <50% toxicity observed for cells treated with the same concentration of free doxorubicin (FIG. 7B). The cell death observed was due primarily to the release and cytotoxic activity of doxorubicin rather than photothermal ablation of the cells by the laser. Irradiation at the highest laser power tested (1.2 W/cm²) for three minutes killed less than 25% of PPC-1 cells treated with doxorubicin-free LTSL-HGN or free HGN at an equivalent concentration (FIG. 7C). In the absence of hollow gold nanoshells, there was minimal heating; irradiation of untreated PPC-1 cells for 10 minutes had no significant impact on cell viability.

Four key elements are conventionally recognized for an effective drug carrier: “retain, evade, target, and release.” (Needham et al., Cancer Res., 2000, 60:1197-201.) Liposomes, including thermosensitive liposomes, have been developed that retain doxorubicin effectively using pH gradient loading. (Allen et al. Adv. Drug Delivery Rev., 2013, 65:36-48; Landon et al., Open Nanomedicine J., 2011, 3:38-64.) Low molecular weight (2000-5000 Da) PEG covalently bound to lipids incorporated into liposome bilayers (PEGylated or “Stealth” liposomes) substantially extend circulation times by allowing the liposomes to “evade” the mononuclear phagocyte system (MPS). Sub-250 nm liposomes that circulate for extended times passively “target” themselves to tumors due, at least in part, to unique features of tumor physiology, which include a high density of abnormally leaky blood vessels and a decreased rate of lymphatic clearance. Together, this combination increases liposome tumor accumulation and is known as the enhanced permeability and retention (EPR) effect. However, retaining drugs, evading the body's defenses, and accumulating in tumors are not enough. The challenge still facing liposomal drug carriers is initiating and controlling drug release when desired, without compromising drug retention.

Tethering hollow gold nanoshells to lysolipid thermosensitive liposomes provides a new method of photothermally triggering local drug release using low intensity, physiologically acceptable near infrared laser light. A lysolipid thermosensitive liposome composition with 15 mol % MPPC, 4 mol % DPSE-PEG²⁰⁰⁰ and 81 mol % DPPC has low permeability to small, charged molecules at 37° C., which provides excellent carboxyfluorescein and doxorubicin retention for 48 hours, similar to other thermosensitive liposome formulations. 40 nm diameter hollow gold nanoshells chemically tethered to the lysolipid thermosensitive liposomes were designed to have a plasmon resonance at 800 nm by controlling the ratio of the shell thickness to shell diameter. The LTSL/HGN constructs range from 100-250 nm in diameter, similar to other liposomes and nanoparticles that show the EPR effect. (Allen et al. Adv. Drug Delivery Rev., 2013, 65:36-48.)

However, each LTSL/HGN carries with it a novel mechanism of near-infrared-light-induced rapid contents release. Irradiating the LTSL/HGN at light intensities an order of magnitude less than intensities previously shown to damage skin provides a sufficient increase in the lysolipid thermosensitive liposome membrane temperature to transiently alter the permeability of the membrane so that the LTSL contents—e.g., drug—can be released without the need to alter the “bulk” temperature of the surroundings. Thus, regional hyperthermia is not required for drug release from the LTSL/HGN. This feature makes the rate of drug release less dependent on the LTSL/HGN concentration; the release rate does not depend on the collective heating of the solution, which is dependent on the hollow gold nanoshell concentration as well as the laser intensity (FIG. 4).

One advantage of using near infrared light to activate LTSL/HGN drug release is that tissue, blood, etc. are relatively transparent to 650-950 nm wavelength light, allowing near infrared transmission in soft tissues at depths up to 10 cm.

Freeing release of the LTSL/HGN contents from regional hyperthermia has nontrivial consequences. The temperature distribution in tissue is difficult to control due to variable thermal conductivity of different tissues—e.g., blood and bone—as well as the natural convective losses due to blood and fluid flow. Temperature increases in off-target tissues must be limited to prevent thermal damage and off-target drug release. In all previous applications of thermosensitive liposomes, regional hyperthermia has been externally applied via bulk heating with water baths, radiofrequency (RF) radiation, microwave (MW) radiation, lasers, and/or ultrasound (Needham et al., Cancer Res., 2000, 60:1197-201; Needham et al., Adv. Drug Delivery Rev., 2001, 53:285-305; Dewhirst et al., Surg. Oncol. Clin. N. Am., 2013, 22:545-61; Stauffer, Int Hyperthermia, 2004, 20:671-677), which often require invasive implants. The rapid interconversion of light energy into heat along with the nanometer scale of the hollow gold nanoshells makes the temperature response extremely rapid, on the order of seconds or less. This makes it feasible to rapidly turn drug release on and off from LTSL/HGNs (FIG. 5), without damaging the ability of the lysolipid thermosensitive liposomes to retain or release drugs (FIG. 6). This allows for the possibility of multiple doses delivered from the same liposomal carriers. The total energy supplied by the laser over the 1-5 minutes necessary for drug release does not result in significant bulk heating. The total energy need only heat the lysolipid thermosensitive liposome bilayer to initiate drug release. Moreover, release only occurs as long as the irradiation continues.

Drug release from the LTSL/HGN with laser heating offers the potential for more effective cell killing than either LTSL/HGN or laser heating alone. (Landon et al., Open Nanomedicine 1, 2011, 3:38-64.) About 90% of PPC-1 cells were killed by doxorubicin released from LTSL/HGN irradiated at 1.2 W/cm² for three minutes compared to about 50% killed by the same concentration of free doxorubicin (FIG. 7C). During irradiation, the sample temperature gradually increased to 41° C. at the end of three minutes of irradiation, and returned to 37° C. within minutes after irradiation ended. Delivery of doxorubicin in combination with mild hyperthermia over the course of hours or days is known to enhance cell killing (Landon et al., Open Nanomedicine 1, 2011, 3:38-64), while direct photothermal ablation may be achieved by increasing the laser power and keeping the cells at high temperatures for extended times. (Zhou et al. J. Am. Chem. Soc., 2010, 132:15351-8; Hirsch et al., PNAS USA, 2003, 100:13549-54; Dickerson et al., Cancer Letters, 2008, 269:57-66; Dreaden et al., Acc. Chem. Res., 2012, 45:1854-65.) However, we observe a near doubling in toxicity for three minutes exposure to a maximum temperature of 41° C., suggesting that the cell membrane permeability towards doxorubicin may be enhanced during this short thermal exposure. Alternatively, the rapid release from the LTSL/HGN may create high local drug concentration gradients in the vicinity of the cells which may result in the enhanced cell killing. Even a temporarily high doxorubicin concentration in the vicinity of the cells may overcome the efflux receptor or other mechanism-driven drug resistance. Without laser induced rapid release, more than 50 times the liposomal doxorubicin concentration is required to induce significant cell toxicity over 48 hours.

Thus, this disclosure describes a novel drug delivery carrier consisting of plasmonic hollow gold nanoshells (HGN) chemically tethered to liposomes made temperature sensitive with lysolipids (LTSL). Continuous-wave irradiation by physiologically acceptable near infra-red light at 800 nm for 2.5 minutes at laser intensities an order of magnitude below that known to damage skin generates heating localized to the liposome membrane. The heating increases the liposome permeability in an irradiation dose-dependent, but reversible manner, resulting in rapid release of small molecules such as the self-quenching dye carboxyfluorescein or the chemotherapeutic doxorubicin. This enables precise spatial and temporal control of contents release with low temperature gradients confined to areas irradiated by the laser focus. The LTSL/HGN separates the mechanism of drug retention from drug release, making it possible to optimize retention and release simultaneously. The LTSL/HGN exhibits a synergistic effect of high local doxorubicin concentrations and local hyperthermia resulting in a near doubling of androgen resistant PPC-1 prostate cancer cell toxicity compared to the same concentration of free doxorubicin.

Thus, more generally, this disclosure describes a composition that includes a liposome and a reversibly heatable component coupled to the liposome. The liposome typically can encapsulate a cargo composition. The compositions described herein permit localized delivery of the cargo composition, even after systemic administration of the liposome composition. The cargo composition can include a pharmaceutical composition and/or a diagnostic composition. A pharmaceutical composition can include, for example, a drug in combination with a pharmaceutically acceptable carrier. In some embodiments, the drug can include an antitumor drug, an antibiotic, an antifungal, or anti-inflammatory drug. In such cases, the liposome composition can provide targeted, localized delivery of the drug at the site of need while decreasing the extent to which the drug is released systemically. This can decrease the amount of drug required to achieve the desired prophylactic and/or therapeutic effect and/or decrease the likelihood and/or extent of undesirable side effects that may result from a more systemic release of the drug. A diagnostic composition can include a compound that carries a detectable label such as, for example, a colorimetric, fluorescent, radioactive, magnetic, or enzymatic label. The gold nanoshell itself can act as a label for X-ray detection.

The liposome generally includes lysolipids and is temperature sensitive. The liposome can include dipalmitoylphosphatidylcholine (DPPC), 1,2-Distearoyl-sn-glycero-3-phosphoethanolamine (DSPE)-PEG²⁰⁰⁰, monopalmitoyl phosphatidyl choline (MPPC), 1,2-Distearoyl-sn-glycero-3-phosphocholine (DSPC), a saturated or unsaturated lysolipid PC, phosphatidylethanolaimines or phosphatidylglycerols with chains of 14-18 carbon (e.g., as an alternative to MPPC), 1,2 Dimyristoyl-sn-glycero-3-phosphocholine (e.g., as an alternative to DSPC, for example, to lower the transition temperature if desired), or any combination of two or more of the foregoing.

In some embodiments, the reversibly heatable component may be coupled to the liposome via a covalent bond. In other embodiments, the reversibly heatable component may be coupled to the liposome via an affinity bond (e.g., avidin-biotin). In other embodiments, the reversibly heatable member may be encapsulated within the liposome itself.

For example, DPPC, DSPE-PEG²⁰⁰⁰-NH₂, DSPE, and MPPC as needed are dissolved in chloroform and the solvent removed by evaporation. The lipids were hydrated overnight at 55° C. with either 50 mM carboxyfluorescein or 300 mM ammonium sulfate, and liposomes were prepared by extrusion, typically by using an Avanti Mini-Extruder with Watson 200 nm polycarbonate filters. Terminal amine groups on the DSPE-PEG²⁰⁰⁰-NH₂ were converted to thiol moieties by adding 2-iminothiolane (Traut's Reagent) at a 1:1 molar ratio of DSPE-PEG²⁰⁰⁰-NH₂:Traut's Reagent and incubating for one hour at 55° C. (FIG. 6).

The reversibly heatable component may be constructed of any suitable material and have any suitable form. In some embodiments, the reversibly heatable component can be in the form of a nanoshell.

The reversibly heatable component may be constructed of any suitable material and have any suitable form. In some embodiments, the reversibly heatable component can be in the form of a nanocube.

In some embodiments, the reversibly heatable component can be in the form of copper sulfide nanoparticles.

In some embodiments, the reversibly heatable component can be tuned to be heated by absorbing radiation having a wavelength within a predetermined window. In some of these embodiments, the radiation may be near infrared—e.g., having a wavelength from about 650 nm to about 950 nm.

In some embodiments, the heatable agent (e.g., either a hollow gold nanoshell, nanocube, or CuS nanoparticle) can be encapsulated within the liposomal drug carrier, ensuring co-localization in a clinical setting and locally increasing the nanoparticle concentration within the liposome. This encapsulated nanoparticle concentration remains constant, even as the liposome sample is diluted, providing sufficient heating from the encapsulated nanoparticles to raise the membrane temperature to the permeability transition temperature of 39-40° C. so as to trigger drug release. We describe a particular example of how to synthesize a thermosensitive liposome with internal heatable elements.

Interdigitated Fusion Vesicles (IFVs) are micron-scale lipid bilayer structures that are effective at encapsulating smaller particles such as metal nanoparticles or vesicles. The IFV is formed by adding ethanol to, for example, saturated DPPC or dipalmitoylphosphatidylglycerol (DPPG) unilamellar vesicles. Ethanol molecules partition between the solution and the bilayer, inducing swelling of the polar headgroups (FIG. 12). The swelling of the DPPC headgroups may make it energetically favorable for the chains to pack side-by-side in an interdigitated configuration as opposed to end-to-end, at least in part by shielding the methyl groups of the adjacent lipid's hydrocarbons chains from the aqueous solution. Interdigitation of the lipid bilayer results in both a decrease in membrane thickness and an increase in membrane rigidity. The imposed curvature stress leads to vesicle rupture as the bending energy exceeds the free energy cost of exposing membrane edges to the aqueous environment. The planar sheets then fuse into larger sheets to lower edge energy, resulting in interdigitated lipid sheets of 1 to 10 microns. The formation interdigitated sheets is readily observable as there is a significant increase in viscosity upon adding the ethanol and the sample changes from a translucent fluid suspension to an opaque milky white gel.

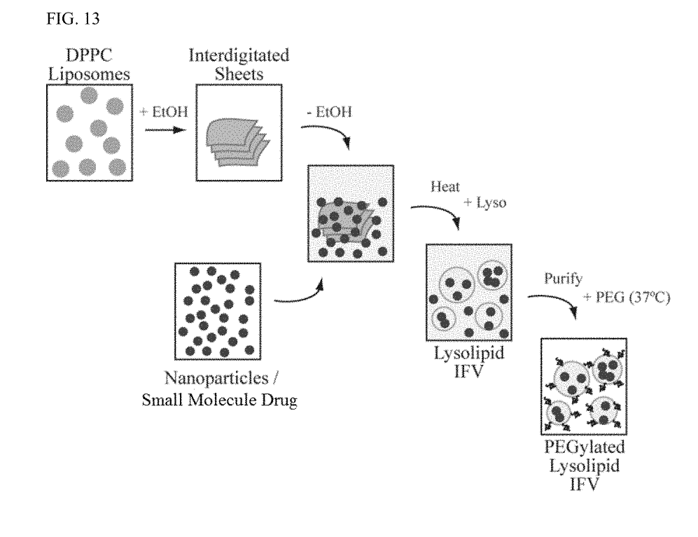

Colloidal particles can be encapsulated during the formation of IFVs from these interdigitated sheets (FIG. 13). First, the ethanol is removed through a series of buffer rinses performed below the transition temperature. X-ray spectra of the DPPC sheets indicate that removing the ethanol causes the lipids to revert from a hexagonal lattice packing, where the lipids are oriented parallel to the membrane normal, to a gel phase with a distorted hexagonal lattice packing, where the lipids are tilted with respect to the membrane normal. (Wong et al., Adv. Materials, 2011, 23:2320-5.) These gel phase sheets remain flat and open as long as the temperature is maintained below the main transition temperature. When the planar lipid sheets are heated above the transition temperature, the hydrocarbon chains melt and the reduced membrane bending energy leads the sheets to bend and formed closed liposomes. Surrounding media and free nanoparticles are passively encapsulated within the interior as the open sheets close into IFVs. IFVs can encapsulate PEGylated CuS nanoparticles or hollow gold nanoshells or nanocubes for heating and triggered release under NIR light irradiation (FIG. 13).

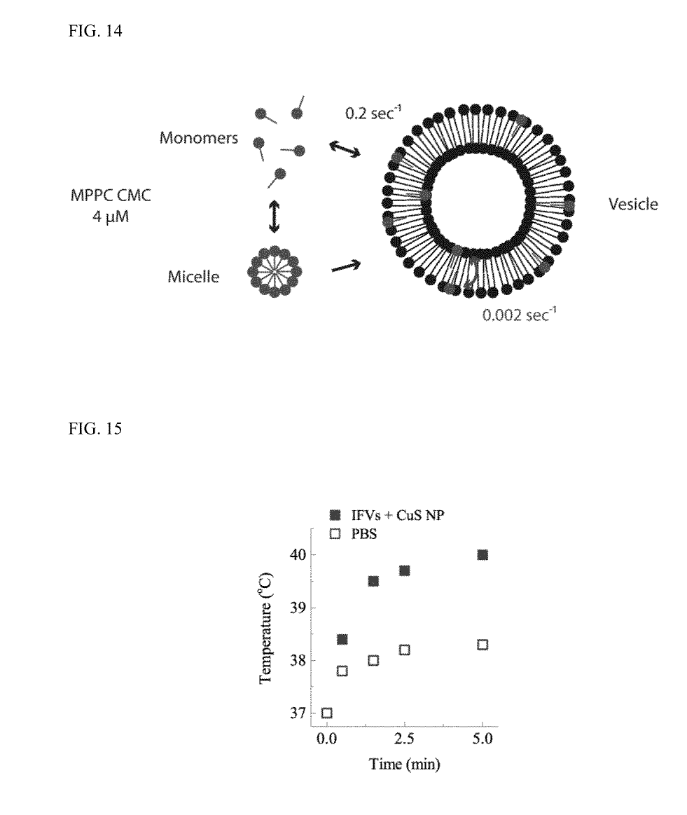

This IFV-formation process typically involves DPPC, DPPG, or mixtures thereof because micelle-forming lipids can interfere with the formation of the interdigitated sheets. Lysolipid and PEGylated lipids can be added to the IFVs after they are formed, however, using a micelle-transfer process. Surfactants with high water solubility such as, for example, single-chained lysolipids or PEG-lipids, can rapidly transfer into the outer leaflet of a fluid phase membrane (FIG. 14). The ability to add lysolipid into pre-formed liposomes and retain the enhanced membrane permeability at the transition temperature enables the formation of lysolipid-containing IFVs. The level of lysolipid within the membrane can be readily controlled by adjusting the concentration of the initial micellar lysolipid added to the sample.

PEG-lipids can include, for example, DSPE-PEG⁵⁰⁰⁰, DSPE-PEG²⁰⁰⁰, DSPE-PEG⁷⁵⁰, etc.; PEG molecules terminated with, for example, NHS, Maleimide, etc.; or Cholesterol PEG.

In some embodiments, the IFVs have a diameter of 100 nm. In other embodiments, the IFVs have a diameter of, for example, 50 nm, 75 nm, 200 nm, 300 nm, 400 nm, 500 nm, 600 nm, 700 nm, 800 nm, 900 nm, 1000 nm, 2000 nm, or 3000 nm, or ranges in between.

In one embodiment, IFVs contain DPPC, MPPC, and copper sulfide nanoparticles. The IFV may further include a PEG-lipid including for example, DSPE-PEG⁵⁰⁰⁰, DSPE-PEG²⁰⁰⁰ or DSPE-PEG⁷⁵⁰, etc. The mol % of MPPC can be about 6, about 7, about 8, about 9, about 10, about 11, about 12, about 13, about 14, about 15, or more than about 15. In some embodiments the mol % of MPPC is less than 20. In one embodiment, the mol % of MPPC is between 8 and 10, in other embodiments, the mol % of MPPC is between 6 and 10, 5 and 10, 4 and 10, 3 and 10, 8 and 12, 8 and 15, 8 and 20, 6 and 12, band 15, or 6 and 20. The mol % of DSPE-PEG²⁰⁰⁰ could be about 1, about 2, about 3, about 4, about 5, about 6, about 7, or more that about 7. In one embodiment, the mol % of DSPE-PEG²⁰⁰⁰ is between 2 and 5, in other embodiments, the mol % of MPPC is between 1 and 5, 2 and 5, 3 and 5, 4 and 5, 1 and 4, 1 and 3, 1 and 2, 2 and 4, or 2 and 6.

Controlling the ratio of lysolipid and, if present, PEG-lipid to DPPC, can be used to tailor the permeability transition temperature of the IFVs and to prevent flocculation or opsonization of the polymer layer. In some embodiments, the combined molar fraction of lysolipid and PEG-lipid to DPPC is 12-15 mol %. In other embodiments, the combined molar fraction of lysolipid and PEG-lipid to DPPC is 10-20 mol %.

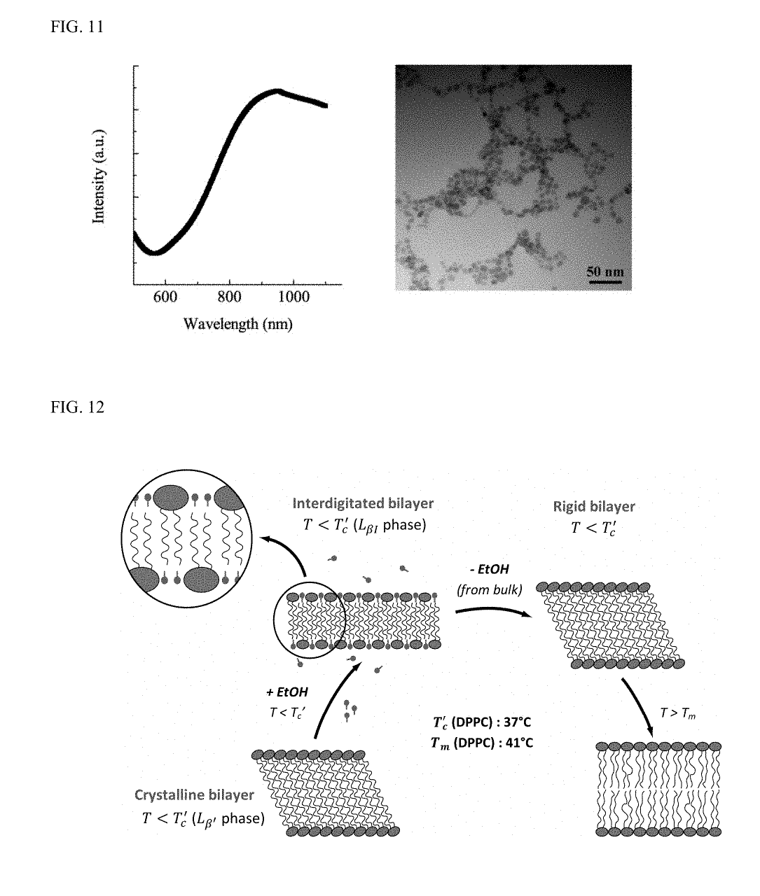

Copper sulfide is a p-type semiconductor that strongly absorbs NIR light; unlike plasmon-resonant gold nanoshells or nanorods, the absorption does not depend on the nanoparticle shape or size. This structure-independent absorption permits the synthesis of smaller diameter nanoparticles, which can be readily encapsulated within a liposome interior. Copper sulfide (CuS) nanoparticles can be encapsulated into DPPC liposomes using the ethanol induced interdigitated phase transition of saturated phospholipids. (Kisak et al., Current Medicinal Chemistry, 2004, 11:199-219; Kisak et al., Langmuir, 2002, 18:284-288.) CuS nanoparticles have a broad absorption peak from 850-100 nm in the NIR (FIG. 11). In some embodiments, copper sulfide nanoparticles have a mean diameter of 5-10 nm, 10-15 nm, 15-20 nm, 20-30 nm, 30-35 nm, 35-40 nm, 40-45 nm, 45-50 nm, 50-55 nm, 55-60 nm, 60-65 nm, 65-70 nm, 75-80 nm, 80-85 nm, 85-90 nm, 90-95 nm, or 95-100 nm. A skilled artisan would recognize that any nanoparticle can be used so long as the nanoparticle is easily incorporated into the IFV. In some embodiments, copper sulfide nanoparticles have a mean size of 9±2 nm.

The concentration of copper sulfide nanoparticles in the IFVs can be altered, depending on the intensity of the NIR to be applied to the target. Higher nanoparticle concentrations require relatively lower intensity NIR to raise the temperature of the IFV to a transition temperature than lower nanoparticle concentrations. Lower nanoparticle concentrations will require higher intensity NIR to raise the temperature of the IFV to a transition temperature than higher nanoparticle concentrations.

In some embodiments, the PEG-lipid in the IFVs creates a layer of polyethylene glycol. The layer of polyethylene glycol can stabilize the liposomes against aggregation and fusion. However, PEG-lipids, have relatively large headgroup to tail group areas also have the potential to destabilize the IFV membrane at concentrations above the overlap concentration The extent of PEG coverage depends both on the molecular weight and the grafting density of the PEG. In some embodiments, the PEG coverage of the IFV is complete. Generally, the higher the molecular weight of the PEG, the lower mol % of PEG that is required for complete coverage. In some embodiments, the PEG lipid is DSPE-PEG⁵⁰⁰⁰ and 2.5-5 mol % provides 100% surface coverage. In some embodiments, the PEG lipid is DSPE-PEG²⁰⁰⁰, and 4-5 mol % provides 100% surface coverage. In some embodiments, the PEG lipid is DSPE-PEG⁷⁵⁰, and approximately 15-18 mol % provides 100% surface coverage.

In some embodiments, the IFVs have a net negative charge. This net negative charge can help minimize aggregation or adsorption to biological surfaces. In some embodiments, the IFVs have a zeta-potential of less than 0 mV, less than −5 mV, less than −8 mV, less than −10 mV, less than −15 mV, less than −20 mV, less than −30 my, less than −35 mV, or less than −40 mV. In some embodiments, the IFVs have a zeta-potential of 0 mV, −8.9 mV, or −39 mV. In some embodiments, the IFVs have a zeta-potential of between 0 mV and −30 mV, 0 mV and −40 mV, between 0 mV and −50 mV, or between 0 mV and −100 mV.

In some embodiments, IFVs are prepared from DPPC liposomes. First, interdigitated DPPC sheets are prepared by adding ethanol to DPPC liposomes. The DPPC sheets are then mixed with copper sulfide nanoparticles and MPPC. The solution is heated to above the transition temperature of the lipid to incorporate the lysolipid within the liposome bilayer and to encapsulate the copper sulfide nanoparticles. After heating, the liposomes can be concentrated and separated from non-encapsulated copper sulfide nanoparticles by rounds of centrifugation and washing.

In some embodiments, PEG-lipid is added to the DPPC liposomes. The interdigitated phase transition can be inhibited if lysolipids or PEG-lipids are added to the DPPC bilayers, so PEG-lipids can be added after the formation of liposomes that encapsulate copper sulfide. In some embodiments, PEG-lipids and liposomes that encapsulate copper sulfide are mixed at 37° C. and the mixture allowed to equilibrate to incorporate PEG-lipid in the liposomes. Excess PEG-lipid can be removed by centrifugation and washing.

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Thermosensitive Liposome Synthesis and Characterization

### Materials

DPPC, DSPE-PEG²⁰⁰⁰-NH₂, DSPE, and MPPC were purchased from Avanti Polar Lipids, Inc. (Alabaster, Ala.). All other reagents were purchased from Sigma-Aldrich (St. Louis, Mo.) unless noted. Carboxyfluorescein 5,6 (CF) was dissolved in sodium hydroxide and the volume was adjusted with 65 mM PBS at pH 7.4 buffer to achieve a final 50 mM carboxyfluorescein solution with physiological osmolarity. Doxorubicin was purchased from Thermo Fisher Scientific (Pittsburgh, Pa) and dissolved at 10 mg/ml in a 10% sucrose solution.

DPPC, DSPE-PEG²⁰⁰⁰-NH₂, DSPE, and MPPC as needed were dissolved in chloroform in glass vials and the solvent removed by evaporation. The lipids were hydrated overnight at 55° C. with either 50 mM carboxyfluorescein or 300 mM ammonium sulfate, and liposomes were prepared by extrusion using an AVANTI Mini-Extruder (Avanti Polar Lipids, Inc., Alabaster, Ala.) with 200 nm polycarbonate filters (Watson Co., Ltd., Kobe, Japan). Terminal amine groups were converted to thiol moieties by adding 2-iminothiolane hydrochloride (Traut's Reagent, Sigma-Aldrich, St. Louis, Mo.) at a 1:1 molar ratio of DSPE-PEG²⁰⁰⁰-NH₂:Traut's Reagent and incubating for one hour at 55° C. For doxorubicin-containing liposomes, a MicroSpin G-50 column (GE Healthcare, Little Chalfont, United Kingdom) was used to exchange external ammonium sulfate for PBS pH 7.4 prior to thiolation and remove excess Traut's Reagent following the reaction. Doxorubicin was added at a 0.05 drug:lipid ratio and incubated overnight at 36° C. to achieve >90% doxorubicin loading. Unencapsulated carboxyfluorescein or doxorubicin was removed by size exclusion chromatography using a PD MiniTrap G-25 column (GE Healthcare, Little Chalfont, United Kingdom) equilibrated with PBS at pH 7.4. Doxorubicin encapsulation was calculated by measuring fluorescence intensity of eluted fractions following liposomal lysis with Triton X-100 using a Varian Cary Eclipse Fluorescence Spectrophotometer (Agilent Technologies, Inc. Santa Clara, Calif.). The lipophilic carbocyanine tracer DiD (Invitrogen, Life Technologies, Corp., Grand Island, N.Y.) at 0.05 mol % was used to identify the liposome-containing fractions, and the doxorubicin fluorescence emission intensity of these fractions was integrated to determine the final doxorubicin concentration.

### Hollow Gold Nanoshells (HGN)

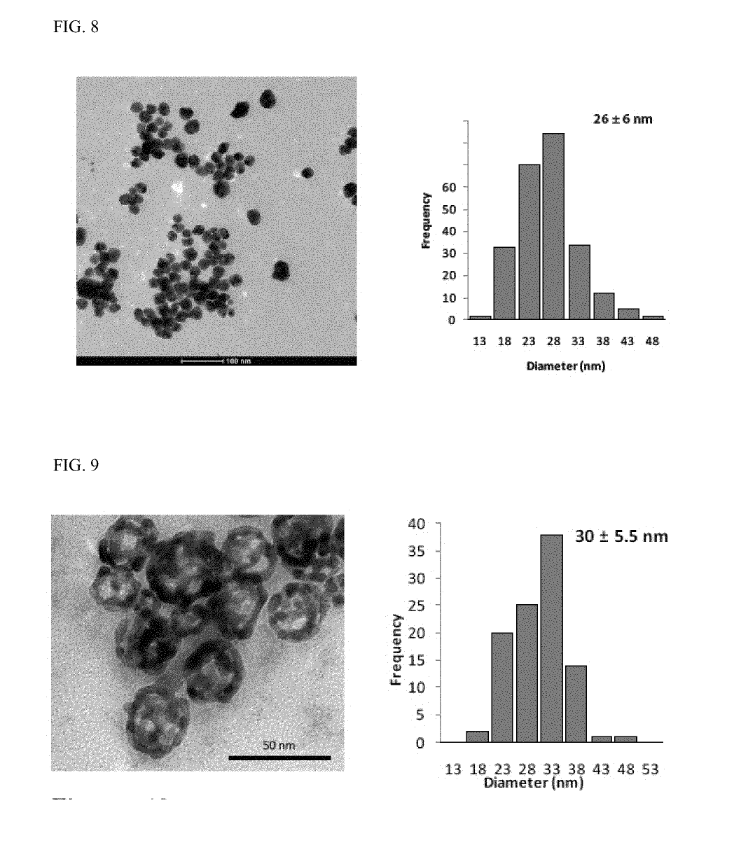

Hollow gold nanoshells were synthesized from solid silver templates using a galvanic replacement reaction by reaction with HAuCl₄. Silver seed particles were prepared by reducing a stirred solution of 500 mL of 0.2 mM AgNO₃ (Sigma-Aldrich, St. Louis, Mo.) and 0.5 mM sodium citrate (Sigma-Aldrich,. St. Louis, Mo.) in deionized water with 0.5 mL of 1.0 M NaBH₄ (EMD) at 60° C. The solution was stirred for two hours and then cooled to room temperature before growing the seed particles to a final target size for use as a sacrificial template for the gold nanoshells growth with the addition of 0.75 mL of 2 M NH₂OH.HCl (Sigma) and 1.75 mL of 0.1 M AgNO₃ and stirred overnight at room temperature (FIG. 8). The galvanic replacement of the silver template particles with gold was optimized to have an absorbance peak at around 800 nm by rapid addition of 3.2 mL of 25 mM Gold III Chloride Hydrate (HAuCl₄, Sigma) at 60° C. (FIG. 9). Silver and gold concentrations were adjusted to center the nanoshell absorbance peak maxima at 800 nm as measured on a Jasco V-530 UV/vis spectrometer (Jasco, Inc., Easton, Md.). Hollow gold nanoshells were stabilized against aggregation in high ionic strength media by adding a surface coating of thiol-terminated methoxypolyethylene glycol (mPEG750-SH, Sigma-Aldrich, St. Louis, Mo.) with a PEG molecular weight of 750 Da. The ratio of gold to mPEG750-NH₂ was optimized to give good stability against aggregation in PBS, while still leaving enough free gold surface to bind to the thiols on the liposomes. The hollow gold nanoshells were washed three times by centrifugation at 12,000×g for 10 minutes and re-dispersed in PBS buffered to pH 7.4. Nanoshells were tethered to the exterior liposome membrane by incubating the PEGylated nanoshells with the liposomes overnight at a 4:1 ratio of nanoshells to liposomes.

### Monodisperse Silver Nanoparticles

Spherical Ag nanoparticles with various sizes were prepared by an optimized polyol method with AgNO₃ as a precursor and diethylene glyocol (DEG) as the solvent. A typical reaction, 0.5 g of polyvinylpyrrolidone (PVP) in diethylene glyocol (DEG, 15 mL) was heated to 180° C. under vigorous stirring over 30 minutes. Then, the temperature was adjusted to and kept at 120° C. Then, 0.10 g of AgNO₃ in DEG (5 mL) was added dropwise to the above solution over two minutes. The reaction was continued for another three minutes at 120° C. The formation of Ag spherical seeds were expressed as a generation of brown/yellow colloidal dispersion. The solution was cooled to room temperature rapidly to quench the reaction.

TABLE 1
Synthesis of Ag spherical seeds.

| | AgNO₃ [g] | PVP [g] | Temp [° C.] | Time* [min] | Size [nm] |
|---|---|---|---|---|---|
| a | 0.10 | 0.50 | 120 | A2R3 | 25 |
| b | 0.10 | 1.50 | 140 | A5R5 | 35 |
| c | 0.10 | 1.50 | 140 | A5R10 | 45 |
| d | 0.10 | 0.50 | 140 | A5R10 | 60 |

*A: the time used to add AgNO₃ dissoloved in 5 mL of DEG, R: the time used to continue the reaction after finishing addition of AgNO₃. Size of the seed is directly proportional to the addition and reaction time.

After the Ag spherical seed mixtures cooled, 1 mL of the solution was vigorously mixed with 9 mL of acetone in a 15 mL centrifuge tube. Then, the tube was centrifuged at 3750 g for 30 minutes. The pellet was collected and dispersed in deionized water (1 mL) in a 1 mL centrifuge tube. The solution was centrifuged at 10×g for 20 minutes. The pellet was dispersed in deionized water (1 mL) and the supernatant was removed. This process was repeated a total of three times. Once each product was consolidated, it was redispersed in 1 mL of deionized water. (FIG. 8).

### Hollow Gold Nanospheres

Hollow gold nanospheres were synthesized by using Ag spherical seeds. 5 mL of Ag seeds were put into 10 mL of beaker and stirred at 500 rpm. 7.5 μL of 2.0 M NH₂OH—HCl and 17.5 μL of 0.1 M AgNO3 were added. Stirring speed was decreased to 200 rpm and the solution was stirred for two hours. Then, the solution was heated to 70° C. and stirred at 360 rpm for 30 minutes, after which the heat was turned off 25 mM of HAuCl₄ was added in 10 μL increments into heated solution with 950 rpm of stirring speed. When each increment was added, UV-Vis spectra were taken to measure the desired absorbance. The addition was continued until the maximum absorbance was observed at around 800 nm of wavelength. A series of color changes were observed during the process. Yellow→orange/red→violet/purple→blue→gray→nearly colorless (FIG. 9).

### Hollow Gold Nanocubes

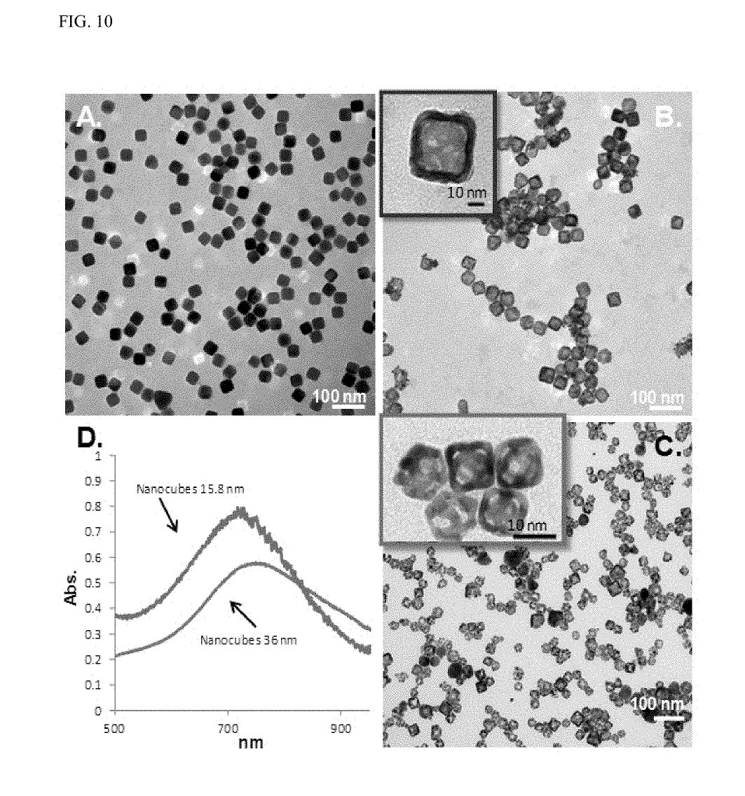

Hollow gold nanocubes of various sizes were synthesized by first preparing Ag nanocubes. Diethylene glycol (DEG, 5 mL) was slowly heated to 150° C. under vigorous stirring (360 rpm) for 30 minutes. Then, 3 mM of sodium hydrosulfide (NaSH) in DEG (60 μL) was added and reacted for four minutes. 3 mM of hydrochloric acid (HCl) in DEG (500 μL) and 1.25 mL of a 20 mg/mL polyvinylpyrrolidone (PVP) solution in DEG were added and reacted for two minutes. 282 mM of silver trifluoroacetate (CF₃COOAg) in DEG (400 μL) was added as the source of Ag. The temperature was kept at 150° C. for the whole process. The reaction time was 30 minutes, 60 minutes, 120 minutes, and 150 minutes, for to produce Ag nanocubes with an edge length of 20 nm, 24 nm, 27 nm, and 34 nm, respectively (FIG. 10).

Hollow gold nanocubes were synthesized using the Ag nanocubes. 500 μL of Ag nanocubes were put into 5 mL of deionized water and stirred at 360 rpm. The solution was heated to 90° C. The reaction can be completed without heat, at 60° C., or at 90° C. The concentration of hollow gold nanocubes increases with temperature because of the increased thermally activated Ag nanocubes. Once the temperature approached 90° C., 0.1 mM of HAuCl₄ was added in 100 μL increments into the heated solution. When each increment was added, UV-Vis spectra was taken to measure the desired absorbance. The addition was continued until the maximum absorbance was observed at around 800 nm of wavelength.

The effective diameters and shell thicknesses of silver nanoparticles, silver nanocubes, hollow gold nanoshells and hollow gold nanocubes were determined via transmission electron microscopy (TEM) using a TECHNAI G2 transmission electron microscope (FEI Co., Hillsboro, Oreg.). All nanoparticle concentrations and averaged size distributions were measured using single particle tracking with a particle-tracking device (NanoSight, Malvern Instruments Ltd., Malvern, United Kingdom).

### Copper Sulfide Nanoparticles

Smaller nanoparticles that do not depend on a hollow core structure may be advantageous in certain circumstances. Copper chalcogenides are well-known p-type semiconductor materials that can act as photothermal agents for biomedical applications. Unlike gold nanoshells or nanocubes, copper sulfide nanoparticles stongly absorb NIR light between 900-950 nm, independent of the nanoparticle structure. This structure-independent absorption enables the synthesis of smaller diameter nanoparticles, which can be readily encapsulated within a liposome interior or within the liposome bilayer. CuS nanoparticles were synthesized in water by stirring 2 mM CuCl₂ with 1.4 mM sodium citrate at room temperature. The pale blue CuCl₂ solution immediately turns a dark brown upon addition of a 2 mM solution of Na₂S. The solution was stirred for five minutes at room temperature followed by 15 minutes at 85-90° C. Reaction completion is indicated by a dark green solution. After cooling the citrate stabilized CuS nanoparticles to room temperature, SH-PEG MW750 was added at a concentration of 1.6 mM overnight to sterically stabilize the nanoparticles. PEGylated CuS nanoparticles were stored at 4° C. The CuS nanoparticles absorb within the near-infrared region, with an absorption peak around 950 nm. Compared to the gold nanoshells, which absorb maximally around 800 nm, CuS absorption occurs at 900-950 nm. The synthesized CuS nanoparticles have a mean diameter less than 10 nm as measured from TEM images (FIG. 11).

Drug Release and Temperature Profiles upon Laser Irradiation

Carboxyfluorescein (CF) release was measured in semi-micro optical glass cuvettes (Starna Scientific Ltd., Essex, United Kingdom) within a custom, temperature-controlled fluorescence spectrometer that was coupled to a continuous wave laser diode for irradiation. 200 μl of sample was placed within a cuvette of 4 mm width and 10 mm path length. The temperature was controlled using a qpod 2e® Peltier sample compartment (Quantum Northwest, Inc., Liberty Lake, Wash.). Irradiation was performed with a continuous wave laser diode with 797 nm wavelength (F6 Series, Coherent Inc., Santa Clara, Calif.) to match the resonance peak maxima of the nanoshells, and the beam diameter was adjusted to 5 mm to ensure that the entire sample volume was irradiated. Incident laser power was varied using an ITC4000 controller (Thorlabs Inc., Newtown, N.J.) and calibrated using a PM30 Optical Power Meter (Thorlabs Inc., Newtown, N.J.). Sample heating was measured with a thermocouple in solution linked to an OMEGAETTE H306 digital thermometer (Omega Engineering, Inc., Stamford, Conn.). At discrete intervals, fluorescence was measured by exciting the sample with a 475 nm LED source (LS-475 Mikropack, Ocean Optics, Inc., Dunedin, Fla.) and measuring the emission spectra with a Maya2000 Pro Spectrometer (Ocean Optics, Inc., Dunedin, Fla.). Fluorescence emission spectra were integrated over the range of 510 to 530 nm, and dye release was calculated as shown in Equation 1:

$$\% Release = \frac{I ( t ) - {I}_{o}}{{I}_{Lysis} - {I}_{o}} , \qquad ( Equation 1 )$$

where I(t) was the intensity at a given time, I₀ was the intensity prior to heating or irradiation, and I_{Lysis} was the intensity accompanying complete release following liposomal lysis with Triton X-100. Release of carboxyfluorescein is shown in FIGS. 2-5.

The androgen resistant human prostate cancer cells (PPC-1) were a generous gift from Erkki Rouslahti (Sanford-Burnham Medical Research Institute, University of California, Santa Barbara, Calif.). They were grown in DMEM/high glucose medium with phenol red (Invitrogen, Life Technologies, Corp., Grand Island, N.Y.), supplemented with 10% FBS (Invitrogen, Life Technologies, Corp., Grand Island, N.Y.). Cells were incubated at 37° C. in 5% CO₂ atmosphere. Cells were plated in 96-well FALCON plates (BD Biosciences, San Jose, Calif.) at 8000 cells per well in 90 μL of medium. After 24 hours, cells were treated with the LTSL/HGN, control LTSL without HGN, or hollow gold nanoshell alone and immediately irradiated with the laser. The continuous wave laser diode (F6 Series, Coherent Inc., Santa Clara, Calif.) was collimated to a beam diameter of 8 mm to irradiate the plate well and incident laser power was adjusted using an ITC4000 controller (Thorlabs Inc., Newtown, N.J.). Irradiation was performed within a temperature controlled chamber to ensure a sample temperature of 37° C. Cell viability was quantified at 5 hours, 24 hours, and 48 hours following irradiation using a resazurin-based assay by adding 10 μl PrestoBlue® (Invitrogen, Life Technologies, Corp., Grand Island, N.Y.) to each well, incubating for 1 hour, and measuring the fluorescence signal on an INFINITE 200 Pro plate reader (Tecan Group Ltd., Switzerland). Each treatment was performed three times with at least four replicates per treatment, and the results were averaged and normalized with respect to the cell-only control. Results are shown in FIG. 7.

### Interdigitation-Fusion Vesicle (IFV) Method

Interdigitated sheets prepared by adding 3 M ethanol to a dispersion of saturated DPPC vesciles were hydrated for one hour at 55° C. with a buffer solution containing CuS nanoparticles and carboxyfluorescein dye for a final concentration of 50 mg/ml DPPC, 4×10¹³ CuS nanoparticles/ml, and 20 mM carboxyfluorescein. Lysolipid was added to the IFV membrane by adding 30 mM micellar MPPC during hydration, which gave an 8 mol % lysolipid fraction within the IFV membrane. Unencapsulated dye, unincorporated lysolipid, and CuS nanoparticle were removed through repeated washes with PBS. Washing was performed with low centrifugal force (1200×g), which caused the IFVs to settle. 10-fold higher centrifugation speeds are required to settle the CuS nanoparticles (the dye and lysolipid do not settle at all).

Repeated exchanges of the supernatant with fresh buffer resulted in a final sample that contained only lysolipid-containing IFV with encapsulated CuS nanoparticle (and encapsulated dye or drug). The lysolipid-containing IFV/CuS nanoparticle were re-suspended in 75 mM PBS at a final concentration of 9 mg/ml DPPC. Given an IFV internal volume fraction of approximately 60% during hydration, the concentration of CuS nanoparticle in the final sample is calculated to be 84 μM CuS [Initial concentration×0.07 dilution×60% encapsulation].

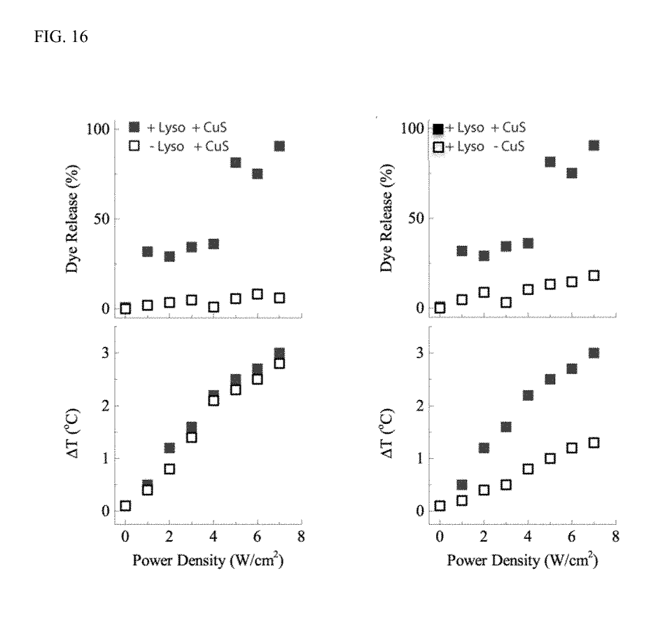

The sample was irradiated at 800 nm within a cuvette controlled at 37° C., and were diluted 80-fold following irradiation to measure fluorescence intensity within the linear regime for carboxyfluorescein. Carboxyfluorescein release was measured as described previously. Under irradiation, the photothermal conversion of the NIR light leads to mild sample heating. Final sample temperature was measured with a thermocouple in solution and the temporal temperature profiles are shown in FIG. 15. After five minutes of irradiation at 7 W/cm², the IFV/CuS sample reached 40° C. while the buffer sample reaches 38° C. FIG. 16 shows that heating by the CuS nanoparticle by five minutes of irradiation with 800 nm NIR light causes almost complete dye release from the lysolipid-containing IFVs. Near-complete dye release is observed from the lysolipid-containing IFVs at laser powers of 5 W/cm² and greater. In contrast, negligible dye is released from pure DPPC IFVs at any laser power. Without CuS nanoparticle, minimal dye is release from lysolipid-containing IFVs because the sample temperature remains below the 39-40° C. membrane transition temperature. With CuS nanoparticle, sample temperature after five minutes of irradiation increases linearly with laser power up to a 3° C. increase (40° C. final sample temperature) at the highest laser power tested of 7 W/cm². Here, release was observed at laser power below the level shown to damage skin using the 800 nm laser. Comparable release may be achieved at lower laser power if the drug delivery carrier were irradiated with longer wavelength light, which would improve the photothermal conversion efficiency of the CuS nanoparticle. At 800 nm, the absorption of the CuS is approximately 40% of the absorption at 950 nm (FIG. 11). Release is enhanced because the encapsulated CuS nanoparticles heat the lysolipid-containing IFV membrane on irradiation with the NIR light, leading to the membrane experiencing a temperature higher than that of the surrounding bulk sample.

### Example 2

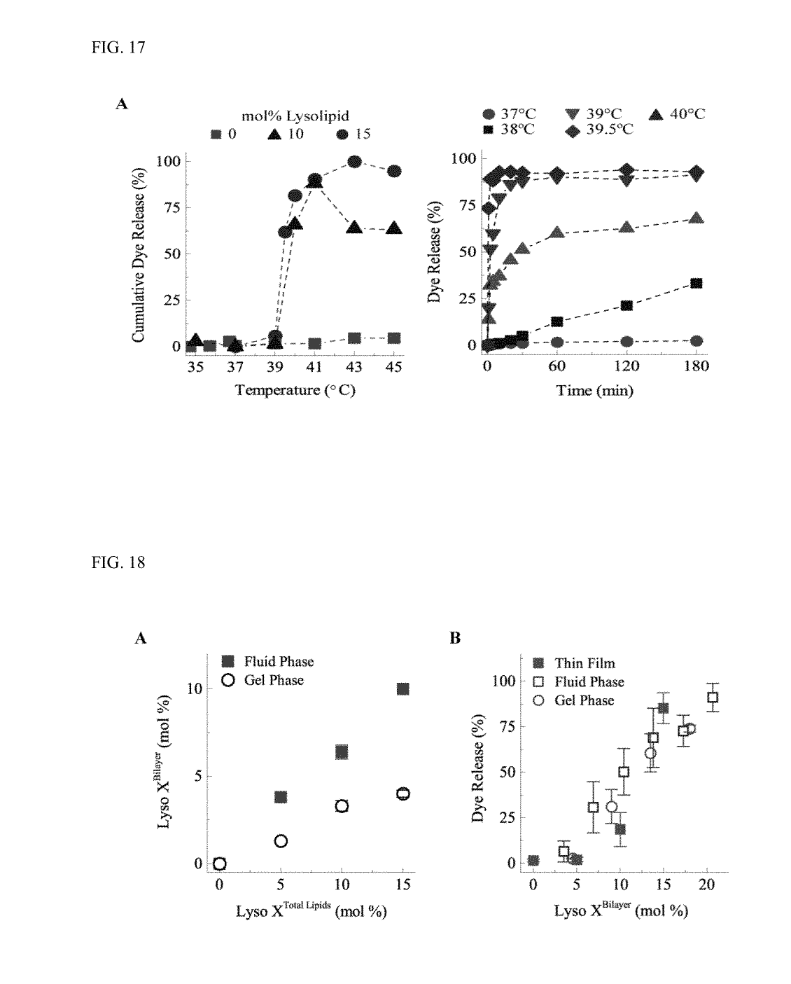

Here, we demonstrate a sequential, self-assembly process to create a composite nanoparticle/interdigitation fusion vesicle (IFV) carrier that uses continuous wave near infra-red (NIR) laser light to initiate and control contents release. Copper sulfide is a p-type semiconductor that strongly absorbs NIR light; unlike plasmon-resonant gold nanoshells or nanorods, the absorption does not depend on the nanoparticle shape or size. This structure-independent absorption enables the synthesis of smaller diameter nanoparticles, which can be readily encapsulated within a liposome interior. 5-10 nm copper sulfide (CuS) nanoparticles can be encapsulated into DPPC liposomes using the ethanol-induced interdigitated phase transition of saturated phospholipids. However, the interdigitated phase transition is inhibited if lysolipids or PEG-lipids are added to the DPPC bilayers, so a second self-assembly step may be utilized. The CuS-DPPC liposomes are made thermosensitive by contacting the liposomes first with a micellar solution of MPPC followed by contact with a micellar solution of PEG-lipid. (N. Forbes et al., Particles and Particle Systems Characterization, 2014, 31:1158-1167.) By controlling the ratio of MPPC and PEG-lipid to DPPC, the liposome membrane composition can be tailored to provide a permeability transition at ˜40° C. (FIG. 17) and a sterically stabilized polymer layer to prevent flocculation or opsonization.

We show that irradiation with low intensity NIR light causes a sufficient temperature rise in the CuS nanoparticles and the liposome membrane to induce the permeability transition and rapidly release the liposome contents. The great advantage of using NIR light to induce release is that tissue, blood, etc. are relatively transparent to 650 nm to 950 nm wavelength light, allowing NIR transmission in soft tissues at depths up to several cm. (Agrawal et al., ACS Nano, 2011, 5:4919-26; Weissleder, Nature Biotechnology, 2001, 19:316-7.) Laser heating induces a near instantaneous response, allowing the liposome contents to be released in seconds. The liposome temperature reverts to ambient quickly when NIR irradiation stops, allowing the liposomes to re-seal which stops drug release. Only lysolipid-containing, thermosensitive CuS-DPPC liposomes irradiated by the laser release their contents, which provides a targeting mechanism for spatial and temporal control of drug release. This inside-outside self-assembly process can be used to encapsulate almost any nanoparticle within a liposome membrane, the composition of which can be modified to include lysolipids for thermosensitivity and PEG-lipids for steric stability.

### Materials

DPPC, methoxy-terminated DSPE-PEG²⁰⁰⁰ and DSPE-PEG⁷⁵⁰, carboxyfluorescein-labeled DPPE-PEG²⁰⁰⁰, and MPPC were purchased from Avanti Polar Lipids (Alabaster, Ala.). The lipophilic carbocyanine lipid (1,1′-Dioctadecyl-3,3,3′,3′-tetramethylindodicarbocyanine perchlorate; DiD) was purchased from Invitrogen and used to label DPPC liposomes as needed. The NBD-labeled lysolipid, 1-{12-[(7-nitro-2-1,3-benzoxadiazol-4-yl)amino]dodecanoyl}-2-hydroxy-sn-glycero-3-phosphocholine, was purchased from Avanti Polar Lipids (Alabaster, Ala.) and used to label the lysolipid fraction. Sodium citrate, copper chloride, sodium sulfide, carboxyfluorescein 5,6 (CF), buffers, solvents and other chemicals were purchased from Sigma-Aldrich Chemical Inc. (St Louis, Mo.) and used as received. The water used in the experiments was of Milli-Q grade with a resistance higher than 18.2 M-ohms-cm.

### Copper Sulfide Nanoparticle Synthesis

CuS nanoparticles were synthesized by stirring 2 mM CuCl₂ with 1.4 mM sodium citrate in water at room temperature. The pale blue CuCl₂ solution immediately turns a dark brown upon addition of an equivalent volume of 2 mM Na₂S. The solution was stirred for 5 minutes at room temperature followed by 15 minutes at 85-90° C. Reaction completion is indicated by the solution turning dark green. After cooling the citrate-stabilized CuS nanoparticles to room temperature, thiol-terminated 750 Da molecular weight polyethylene glycol (SH-PEG MW750) was added at a concentration of 1.6 mM and stirred overnight at room temperature to coat the nanoparticles with PEG to stabilize the CuS against flocculation and sedimentation. PEGylated CuS nanoparticles were stored at 4° C. until use. CuS absorbance was measured using a Jasco V-530 UV-vis spectrometer and the size distribution determined by conventional transmission electron microscopy imaging after spreading the CuS nanoparticles on formvar-covered TEM grids and drying.

### Interdigitation-Fusion Vesicles

DPPC was dissolved in chloroform in glass vials and the solvent removed by evaporation. If needed, 0.1-1 mol % diD dye could be added to the DPPC in chloroform. The lipid was hydrated overnight at 55° C. in PBS at 25 mg/ml DPPC. 50-100 nm diameter unilamellar liposomes were prepared by performing at least five freeze-thaw cycles, followed by extrusion in an Avanti Mini-Extruder (Avanti Polar Lipids, Alabaster, Ala.) using 100 nm pore diameter filters. The DPPC (or modified DPPC) liposomes were transformed into interdigitated bilayer sheets by dropwise addition of ethanol (3 M net ethanol concentration) to the liposome suspension at room temperature (Boyer et al., ACS Nano, 2007, 1:176-182; Ahl et al., Methods in Enzymology, 2003, 367:80-98). The interdigitated sheets were centrifuged at low speed to pellet the sheets, and then washed with buffer. Carboxyfluorescein 5,6 (CF) was dissolved in sodium hydroxide and the volume was adjusted with 65 mM PBS at pH 7.4 buffer to achieve a final 50 mM CF solution with physiological osmolarity. PEGylated CuS nanoparticles were mixed with the CF solution and added to the interdigitated sheets and the solution was held at 55° C. for 20 minutes to induce encapsulation of the CuS nanoparticles and CF and form closed liposomes. If needed, 30 mM MPPC in buffer was added to incorporate lysolipid into the liposomes during the heating process; the desired ratio of MPPC to DPPC in the final liposomes was set by the mole ratio of MPPC:DPPC in solution. Liposomes were separated from unencapsulated CuS nanoparticles and unincorporated MPPC by repeated slow speed centrifugation followed by exchange of the supernatant with fresh PBS. To sterically stabilize the liposomes, DSPE-PEG²⁰⁰⁰ was added to the solution at 5 mol % of the total liposome lipid concentration at 37° C. and the mixture allowed to equilibrate for 48 hours. Excess DSPE-PEG²⁰⁰⁰ was removed by centrifugation and repeated washing with buffer. The average size of the liposomes was determined using cryo-TEM imaging as described below or using single-particle tracking with a Nanosight NTA 2.3 particle-tracking device.

### NIR Irradiation and Dye Release

CF release from the liposomes was measured in semi-micro optical glass cuvettes (Starna Scientific Ltd., Essex, United Kingdom) within a custom, temperature-controlled fluorescence spectrometer that was coupled to a continuous wave laser diode for irradiation. 200 μl of sample was placed within a cuvette of 4 mm width and 10 mm path length. The temperature was controlled using a qpod 2e® Peltier sample compartment (Quantum Northwest, Liberty Lake, Wash.). Irradiation was performed with a continuous wave laser diode at 797 nm (F6 Series, Coherent Inc., Santa Clara, Calif.) with the beam diameter adjusted to 5 mm to ensure that the entire sample volume was irradiated. The incident laser power was varied using an ITC4000 controller (ThorLabs Inc., Newtown, N.J.) and calibrated using a PM30 Optical Power Meter (ThorLabs Inc., Newtown, N.J.). Sample heating was measured with a thermocouple in solution linked to an Omegaette H306 digital thermometer (Omega Engineering, Stamford, Conn.). At discrete intervals, fluorescence was measured by exciting the sample with a 475 nm LED source (LS-475 Mikropack, Ocean Optics, Inc., Dunedin, FL) and measuring the emission spectra with a Maya2000 Pro Spectrometer (Ocean Optics, Inc., Dunedin, Fla.). Fluorescence emission spectra were integrated over the range of 510 nm to 530 nm, and dye release was calculated according to Equation 1, as described in Example 1.

### Zeta Potential Measurements

A Malvern ZetaSizer Nano ZS (Westborough, Mass.) instrument was used to determine zeta potentials. About 750 μl of sample liquid was deposited into the sample cuvette. A laser beam within the instrument is split to provide a reference and incident beam. The incident beam passes through the center of the sample cell and the scattered light at an angle of about 13° is detected. An electric field of optimal intensity determined by the instrument software is applied to the cell and the particle movement causes the intensity of light to fluctuate with a frequency proportional to the particle speed. This information is passed to a digital signal processor and then to a computer to produce a frequency spectrum from which the electrophoretic mobility and zeta potential are calculated.

### TEM Characterization

Aqueous suspensions were spread as a thin film (0.5-10 μm) onto formvar-coated electron microscopy grids (SPI Supplies, West Chester, Pa.) within a Vitrobot Mark IV (FEI, Hillsboro, Oreg.) to ensure a reproducible sample thickness, minimal sample evaporation prior to cooling (and potential concentration or reorganization of the sample), and an optimal cooling rate. Following equilibration, the samples were rapidly plunged into liquid ethane cooled in a bath of liquid nitrogen. After vitrification, samples remain submerged under liquid nitrogen until transfer via a GATAN (Pleasanton, Calif.) cryo-transfer unit to a FEI Technai Sphera G2 transmission electron microscope to maintain sample temperature below −170° C. “Low-Dose” imaging conditions were used to prevent sample disruption due to melting, chemical reactions and other forms of radiation damage (Coldren et al., Langmuir, 2003, 19:5632-5639).

### Lysolipid/PEG Partitioning

Unilamellar liposomes were synthesized at 25 mg/ml using the thin film hydration technique and then extruded to 100 nm in diameter as described above. A red carbocyanine membrane dye (DiD) was included to track the liposome population. A 30 mM micellar solution of lysolipid was prepared by hydrating a dried film of lysolipid with PBS. The micellar solution contained 10 mol % NBD-labeled lysolipid (green). To track PEG partitioning, 2.5 mol % of fluorescently labeled DSPE-PEG²⁰⁰⁰ was added to DSPE-PEG²⁰⁰⁰. An aliquot of the appropriate labeled micellar solution was incubated overnight (18-20 hours) with the labeled liposomes at either 37° C. or 55° C. Uptake of the lysolipid by the gel or liquid crystalline phase membrane was assessed by isolating the liposome fraction using a gravity size exclusion column and quantifying and comparing the red liposome and green lysolipid fluorescence signals of the elution fractions. The much larger liposomes (50-100 nm) elute more rapidly from the column than the smaller micellar (5 nm) or monomeric lysolipid or DSPE-PEG²⁰⁰⁰.

### Results and Discussion

Thiol-PEG stabilized CuS nanoparticles absorb strongly in the near-infrared region, with a broad absorption peak from 800 nm to 1000 nm (FIG. 11). The synthesized CuS nanoparticles are small, with mean diameter less than 10 nm as measured from TEM images. A concentration of 10¹⁵ nanoparticles/ml was calculated based on the average size determined from TEM, assuming complete reaction.

Efficient encapsulation of the CuS nanoparticles into liposomes was done by taking advantage of the interdigitated phase of DPPC and similar saturated phospholipids (FIG. 12). Cooling L_{α} phase dipalmitoylphosphatidylcholine (DPPC) liposomes from above the gel-liquid crystal temperature, T_{c}, of 41° C. to room temperature causes the acyl chains of the lipids to crystallize and tilt to accommodate the area mismatch between the phosphocholine headgroups and the acyl chains, leading to the L_{β′} or gel phase (FIG. 12). Adding 3 M ethanol to the L_{βI′} phase swells the headgroup region, further increasing the area mismatch between the headgroups and acyl chains, resulting in the interdigitated L_{βI} phase. Wide angle X-ray diffraction of the interdigitated sheets shows a single reflection at q≈15.3 nm⁻¹ indicating an untilted hexagonal lattice with d≈0.41 nm, consistent with the interdigitated L_{βI} phase. Interdigitation of the lipid bilayer results in both a decrease in membrane thickness and an increase in membrane rigidity. Small (100 nm) unilamellar vesicles rupture in the L_{βI} phase as the bending energy exceeds the free energy cost of exposing membrane edges to the aqueous environment. The resulting open bilayer sheets then fuse into larger sheets to lower edge energy, resulting in interdigitated lipid sheets of 1 μm to 10 μm in extent. The transition to interdigitated sheets is accompanied by a significant increase in viscosity and the liposome suspension changes from a translucent fluid to an opaque, milky-white gel.

The open stacks of bilayers remain after the replacement of the ethanol-aqueous buffer mixture with pure buffer as long as T<T_{c}. However, X-ray diffraction shows that the bilayer structure changes; the single reflection of the interdigitated phase separates into a sharp reflection at q≈14.8 nm⁻¹ with a broad shoulder at q 15.25 nm⁻¹ consistent with the tilted L_{β′} phase. When the planar lipid sheets are heated above 41° C. into the liquid crystalline, or L_{α} phase, the hydrocarbon chains melt and the reduced membrane bending energy makes closed liposomes the minimal energy state. In the process of forming closed liposomes, CuS nanoparticles suspended with the bilayer sheets (or any nanometer scale particles in the suspension) are encapsulated. On cooling to room temperature, the bilayers re-enter the L_{β′} phase, but the liposomes remain closed and retain their contents. This metastable phase progression can accommodate small fractions (˜3-5 mol %) of fluorescently labeled lipids or cholesterol, or larger fractions of saturated dipalmitoylphosphatidylglycerol in the final liposomes if needed.

However, this interdigitation-fusion process cannot accommodate lysolipids and PEG-lipids at the mole fractions necessary to promote rapid permeability changes (FIG. 17) and steric stabilization. Interdigitation has been observed for DPPC bilayers with low levels (<5 mol %) of 750 Da molecular weight PEG-lipids, but lysolipid and DSPE-PEG²⁰⁰⁰ at the necessary combined molar fraction of 12-15 mol % prevent the interdigitation transition (B. Wong et al., Adv. Materials, 2011, 23:2320-2325).

The necessary lysolipid and PEG-lipid fractions can be added to the CuS liposomes by the spontaneous partitioning of micellar lysolipid and PEG-lipid into the bilayer. MPPC and DSPE-PEG²⁰⁰⁰ are relatively soluble in aqueous solution and form micelles that can rapidly exchange and partition into the liposome bilayer. We explored the rate and extent of equilibrium partitioning of MPPC and DSPE-PEG²⁰⁰⁰ micelles into interdigitation-fusion liposomes. Partitioning into the membrane below the permeability transition temperature is preferred to minimize leakage of encapsulated small molecules at the phase transition temperature (FIG. 17) and may be advantageous for incorporating temperature sensitive biological ligands attached to the PEG-lipids. Fluorescently labeled lysolipid and PEG-lipids were used to evaluate the uptake of lysolipid and PEG-lipid from a micellar solution into pre-formed fluorescently labeled liposomes. Release of encapsulated CF was used to determine the impact on permeability and determine the limits of liposome stability.

FIG. 14 shows a schematic of lysolipid (or DSPE-PEG²⁰⁰⁰) insertion into a liposome bilayer. In the external solution, the lysolipid exists both in micelles and in its monomeric form at its critical micelle concentration (CMC, 4 μM for MPPC). Lysolipid monomers rapidly diffuse throughout the solution and partition into the liposome bilayer. Adsorption of MPPC monomers into the outer bilayer leaflet occurs at a rate of 0.2 sec⁻¹ as measured using micropipette techniques. (Needham et al., Biophys. J., 1997, 73:2615-2629; Needham et al., Ann. Biomed. Eng., 1995, 23:287-298.) Lysolipid micelles can also fuse with the membrane, but this process happens more slowly due to the larger size of the micelles relative to the monomers; the micelles primarily act as a depot to keep the monomer concentration at the CMC. As the lysolipid partitions into the outer bilayer leaflet, the unequal distribution between the outer and inner leaflet leads to an increase in the surface area of the outer monolayer relative to the inner monolayer. The area per molecule of the inner monolayer necessarily must increase to match the outer monolayer, creating tension across the membrane, and defects that promote the exchange (flip-flop) of lysolipid across the membrane.

Unilamellar DPPC liposomes of 100 nm diameter at 25 mg/ml with 0.1 mol % of the red carbocyanine membrane dye, diD, were mixed with various amounts of a 30 mM micellar solution of MPPC lysolipid in PBS. The MPPC was labeled with 10 mol % of green NBD-MPPC analog; our results are consistent with the NBD-lysolipid partitioning in a similar fashion as the unlabeled lysolipid (as determined by CF release as a function of temperature, FIG. 18). The mixtures were incubated either one hour or overnight (18-20 hours) at 37° C. (gel phase, below T_{c}) or 55° C. (fluid phase, above T_{c}). The partitioning of the lysolipid between gel or fluid phase liposomes and micelles was determined by separating the liposomes and micelles using a gravity size exclusion column followed by quantifying the relative DiD and NBD fluorescence signals of the elution fractions corresponding to the liposomes. The liposomes, being an order of magnitude larger than the micelles, eluted first.

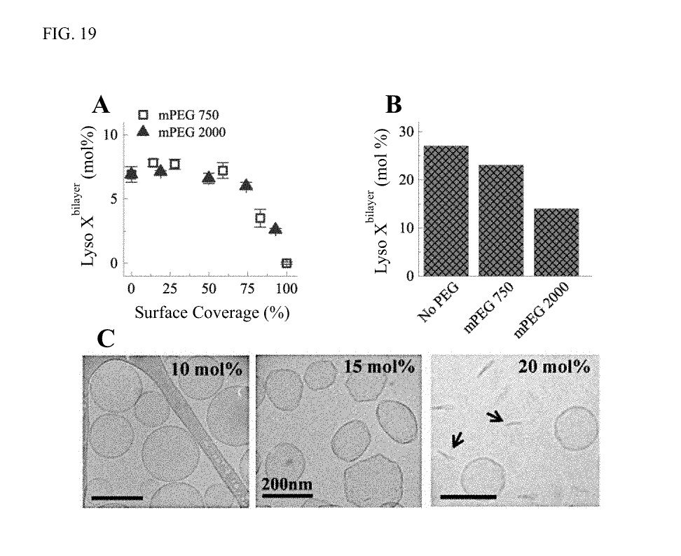

MPPC partitions into both the low temperature gel or the high temperature fluid phase DPPC bilayers, although the partitioning was twice as great for the high temperature, fluid L_{α} phase (FIG. 18A). Membrane uptake (Lyso X^{Bilayer}) was proportional to the concentration of MPPC in solution (Lyso X^{Total Lipids}) relative to DPPC. Equilibrium partitioning was reached in one hour, as samples incubated overnight had negligible additional uptake. To evaluate the effect of the lysolipid on the permeability, the bulk sample temperature was rapidly increased to 40° C. and held for 2.5 minutes. No difference in fractional dye release and hence, membrane permeability, was observed between liposomes made with the lysolipid present in the initial lipid mixture (Thin Film in FIG. 18B) versus liposomes that had lysolipid added from micellar solution following formation either at low or high temperature. Adding lysolipid into interdigitation-fusion liposomes allows for the same enhanced membrane permeability at the transition temperature, making the interdigitation-fusion liposomes thermosensitive. We determined that DPPC interdigitation-fusion liposomes with an MPPC fraction of 8-10 mol % in combination with 4 mol % DSPE-PEG²⁰⁰⁰ provides an combination of fast content release on heating and long-term stability prior to heating. Liposomes destabilized, that is, were not capable of retaining internalized CF, at MPPC concentrations exceeding 20 mol %. FIG. 19C shows that DPPC forms lysolipid-stabilized bilayer discs coexisting with ruptured liposomes at higher MPPC mole fractions. (N. Forbes et al., Particles and Particle Systems Characterization, 2014, 31:1158-1167.)

Liposomes can incorporate a layer of polyethylene glycol to stabilize the liposomes against aggregation and fusion. When tethered to a surface at low grafting density, the hydrophilic PEG polymer chains extend into the aqueous solution in a random coil conformation in the “mushroom” regime. As the grafting density increases, the PEG chains repel each other laterally, causing the PEG polymer chains to elongate and extend further into the solution, forming an extended polymer “brush” configuration (S. Zalipskyet al., J. Controlled Release, 1996, 39:153-161; A. L. Klibanov et al., FEBS Letters, 1990, 268:235-237). The extent of PEG surface coverage depends both on the molecular weight and the grafting density of the PEG. The radius of gyration of a PEG chain in water scales as:

R_{F}˜lₛN^{3/5} (Equation 2)

in which lₛ is the length of the polymer monomer (0.35 nm for the ethylene oxide repeat units in PEG) and N is the number of monomers in each PEG chain. DSPE-PEG²⁰⁰⁰ has 45 ethylene-oxide monomers, so R_{F}˜3.5 nm, while DSPE-PEG⁷⁵⁰ has 17 monomers giving an R_{F}˜2 nm. (A. K. Kenworthy et al., Biophys. J., 1995, 68:1921-1936.) The PEG-lipid headgroup area is proportional to the length of the PEG chain according to:

$$A \text{∼} {π ( \frac{1}{2} {R}_{F} )}^{2} \qquad ( Equation 3 )$$

giving a headgroup area of 9 nm² for DSPE-PEG2000 and 3 nm² for DSPE-PEG⁷⁵⁰. The percentage of PEG-lipids leading to complete surface coverage of random coils can be estimated from the ratio of the area of the DPPC headgroup (˜0.5 nm²) to the area of the PEG-lipid headgroup (Equation 3). This estimate gives the transition coverage from the mushroom to brush regime at ˜5 mol % DSPE-PEG²⁰⁰⁰ and ˜17 mol % DSPE-PEG⁷⁵⁰. Clinically used Doxil (liposomal doxorubicin), contains ˜4 mol % of DSPE-PEG²⁰⁰⁰ (T. M. Allen, Current Opinion in Colloid and Interface Science, 1996, 1:645-651) so steric stabilization and prevention of opsonization and clearance in the circulation correlates with a near-complete mushroom concentration in which the liposome surface is covered by PEG.

FIG. 19A shows that DSPE-PEG on the bilayer also inhibits lysolipid transfer into the bilayer. 100 nm unilamellar DPPC liposomes with various mole fractions of either DSPE-PEG²⁰⁰⁰ or DSPE-PEG⁷⁵⁰ were mixed with dye-labeled MPPC micellar solutions at 10 mol % of the total DPPC concentration. A 100% surface coverage for DSPE-PEG²⁰⁰⁰ was 5 mol % and 17 mol % for DSPE-PEG⁷⁵⁰. FIG. 19A shows that PEG surface coverage exceeding 50% reduces lysolipid transfer into the bilayer; 100% surface coverage prevents any lysolipid incorporation into the liposomes. No difference was observed between DSPE-PEG²⁰⁰⁰ liposomes and DSPE-PEG⁷⁵⁰ liposomes at equal surface coverage despite the difference in the radius of gyration for these two molecular weights. This suggests that lysolipid accesses the bilayer through gaps between PEG molecules on the surface, as the thickness of the PEG coating is determined by the molecular weight while the surface coverage determines the extent of free space between the polymer chains.

PEG-lipids, similar to lysolipids, have relatively large headgroup to tail group areas, form micelles in aqueous solution, and also have the potential to destabilize the liposome membrane at concentrations above the overlap concentration. FIG. 19B shows that the total mole fraction of lysolipid plus PEG-lipids within the bilayer determines liposome stability. Liposome stability was assessed by measuring CF retention; a complete lack of CF retention was taken to be an indication of liposome destabilization. In the absence of PEG-lipid, liposomes remained stable up to 25 mol % MPPC. 50% surface coverage of DSPE-PEG⁷⁵⁰ slightly reduced the total amount of MPPC that could be incorporated prior to destabilization. However, 50% surface coverage of DSPE-PEG²⁰⁰⁰ reduced the amount of MPPC to <15 mol % for stable liposomes.

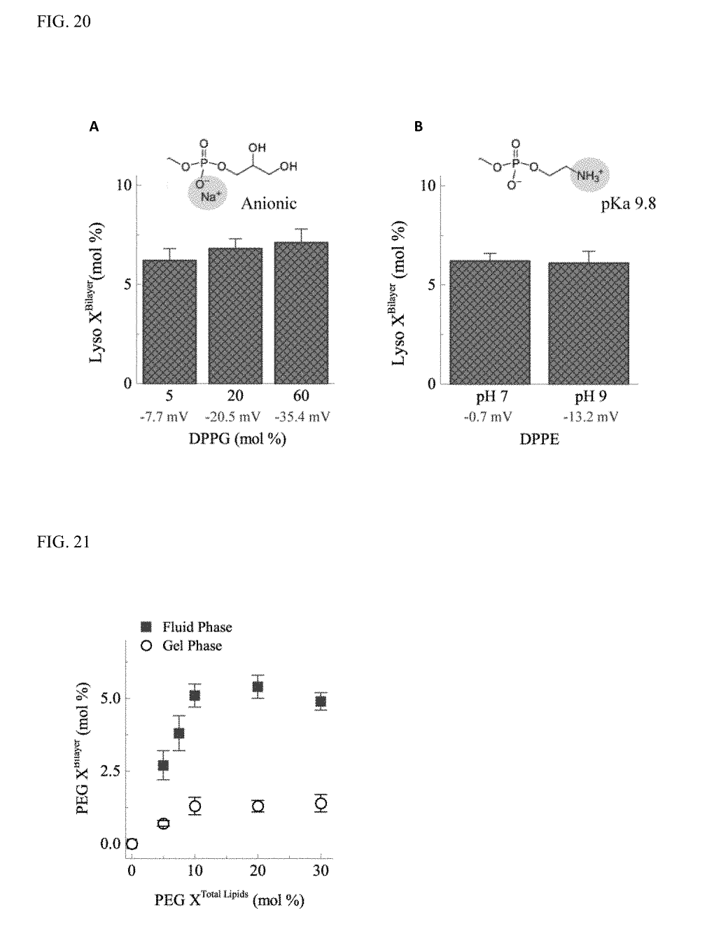

In addition to a PEG-lipid coating, it may be useful to add a net negative charge to the liposomes to minimize aggregation or adsorption to biological surfaces, which are predominantly negatively charged. The DSPE-PEG²⁰⁰⁰ used here is terminated with an anionic methoxy group; liposomes with 5 mol % of methoxy-terminated DSPE-PEG²⁰⁰⁰ have a zeta-potential of −8.9 mV. In FIG. 20, DPPC liposomes with various mole fractions of dipalmitoylphosphatidylglycerol (DPPG) were compared to confirm that the steric effect of DSPE-PEG²⁰⁰⁰, rather than the negative surface charge, impact lysolipid uptake into the membrane. The inset of FIG. 20A shows the negatively charged phosphatidylglycerol headgroup. The zeta potential of DPPC liposomes is ˜0 mV in the absence of DPPG but decreases to ˜35 mV with 60 mol % DPPG. However, within experimental error, the surface charge of the liposomes did not change the partitioning of MPPC into the bilayer.

The headgroup charge of dipalmitoylphosphatidylethanolamine (DPPE) varies with pH over the range of pH 6 to pH 10. The DPPE headgroup contains a terminal amine group with a pKa of 9.8. The amine group is protonated and the headgroup is net neutral at pH 7; increasing the pH leads to deprotonation of the headgroup and a negative surface charge. MPPC added to DPPC:DPPE liposomes at either pH 7 or pH 9 did not alter lysolipid partitioning over the range of zeta potentials and pH that might be encountered for typical liposome formulations.

Although DSPE-PEG²⁰⁰⁰ has two fully saturated stearoyl chains, its large hydrophilic headgroup leads to its self-assembly into micelles (CMC=5.8 μM) in aqueous solutions, similar to the lysolipids. (P. S. Uster et al., FEBS Letters, 1996, 386:243-246; S. Zalipsky, et al., J. Controlled Release, 1996, 39:153-161; S. Zalipskyet al., FEBS Letters, 1994, 353:71-74.) A 15 mM micellar solution of DSPE-PEG²⁰⁰⁰ with 2.5 mol % of fluorescently labeled DSPE-PEG²⁰⁰⁰ was added to a suspension of 40 mM, 200 nm diameter DPPC liposomes. Following separation of the liposomes in a gravity size exclusion column, the relative liposome and DSPE-PEG²⁰⁰⁰ fluorescence were compared. FIG. 21 shows that the DSPE-PEG²⁰⁰⁰ transfers into both fluid and gel phase lipid bilayers, similar to the lysolipids. Transfer into the fluid phase membrane was more rapid than into the gel phase bilayer. In contrast to lysolipid uptake, which reached equilibrium within an hour, complete uptake of PEG-lipid occurred more slowly. Within the fluid phase, about 50% of the DSPE-PEG²⁰⁰⁰ transferred into the membrane within the first hour, however, DSPE-PEG²⁰⁰⁰ continued to partition into the membrane for 48 hours. In the fluid phase, liposome bilayer concentrations exceeding 5 mol % DSPE-PEG²⁰⁰⁰ led to liposome destabilization, as measured by release of CF from the liposomes. In the gel phase, the liposomes remained stable at 5 mol % DSPE-PEG²⁰⁰⁰. A final concentration of 4-5 mol % DSPE-PEG²⁰⁰⁰ can be achieved within ˜1 hour at 55° C. in the fluid phase or after 48 hours at 37° C. with an initial DSPE-PEG²⁰⁰⁰ concentration of 10 mol % of the total lipids (data not shown).

### Interdigitation-Fusion Vesicle Construction

From our preliminary work, thermosensitive, sterically stabilized interdigitation-fusion liposomes can contain 8-10 mol % MPPC and 4 mol % DSPE-PEG²⁰⁰⁰, with the remainder being DPPC. FIG. 13 shows a schematic of the self-assembly process we used to make lysolipid-containing thermosensitive vesicles with encapsulated CuS nanoparticles stabilized by DSPE-PEG²⁰⁰⁰. Interdigitated DPPC sheets were prepared by adding 3M ethanol to extruded 50 nm DPPC liposomes in PBS buffer at room temperature, which converted the translucent blue liposome suspension into an opaque, milky-white gel. The interdigitated sheets were centrifuged at low speed to concentrate the sheets and washed repeatedly with fresh buffer to remove the ethanol. After washing, the pellet of DPPC sheets were mixed with an aqueous suspension of freshly prepared, thiol-PEG stabilized CuS nanoparticles (10¹⁵ CuS NP/ml) and carboxyfluorescein dye (as needed) for a final concentration of 50 mg/ml DPPC, 0.41×10¹⁵ CuS NP/ml, and 20 mM CF. Sufficient 30 mM micellar MPPC was added to reach a solution ratio of about 6:1 DPPC:MPPC (see FIG. 18A), and the mixture was heated for 1 hour at 55° C. to incorporate 8-10 mol % lysolipid within the liposome bilayer. After heating, the lysolipid-containing, CuS encapsulated liposomes were concentrated at low centrifugal force (1200×g) and the supernatant exchanged for buffer. Approximately 10-fold higher centrifugation speeds are required to sediment the PEGylated CuS NP. By repeating the centrifugation and washing, the final sample contained only the lysolipid-DPPC liposomes with encapsulated CuS NP and CF.

Following purification, CuS NP liposomes were re-suspended in 75 mM PBS at a final concentration of 9 mg/ml DPPC. Assuming a liposome internal volume fraction of 60%, the concentration of CuS NP in the final sample is [Initial concentration×0.07 dilution×60% encapsulation] or ˜80 μM CuS. To sterically stabilize the liposomes, DSPE-PEG²⁰⁰⁰ was added at 5 mol % of the total liposome lipid concentration at 37° C. and the mixture allowed to equilibrate for 48 hours. Excess DSPE-PEG²⁰⁰⁰ was removed by centrifugation and repeated washing with buffer. Introduction of DSPE-PEG²⁰⁰⁰ as the final stage of the process simplifies purification during the synthesis as the PEGylated liposomes sediment much more slowly than non-PEGylated liposomes.

### NIR-Triggered Dye Release

The interdigitation-fusion liposomes (IDL) contained CuS nanoparticles within the liposome interiors with MPPC and DSPE-PEG²⁰⁰⁰ incorporated into the bilayer membrane by self-assembly. The IDL were irradiated by continuous, 800 nm NIR light within a cuvette controlled at 37° C. The absorption and photothermal conversion of the NIR light by the CuS nanoparticles in the IDL leads to an increase in the sample temperature as measured with a thermocouple (FIG. 15). After five minutes of irradiation at 7 W/cm², the liposomes with encapsulated CuS reached 40° C. (ΔT=3° C.), while the buffer only reached 38° C. (ΔT=1° C.). This is consistent with the strong absorption of the CuS and the relatively weak absorption by water at 800 nm. Lower power densities would be required for a NIR light source in the range of 900-950 nm, over which CuS has more than twice the specific absorption (FIG. 11).

Both encapsulated CuS nanoparticles and MPPC in the liposome bilayer are required for rapid contents release (FIG. 16). Heating under NIR light irradiation initiates near-complete dye release from the lysolipid-containing IDL at laser power densities ≧5 W/cm² within five minutes. In contrast, negligible dye is released from DPPC or DPPC plus MPPC IDL at any laser power used without encapsulated CuS. Without the specific absorption of NIR light by the CuS NP, the sample temperature remains below the ˜40° C. membrane transition temperature, leading to minimal dye release. With CuS laser power up to a 3° C. increase (40° C. final sample temperature) at the highest laser power tested of 7 W/cm². This power intensity is well below the 12 W/cm² threshold observed in previous studies that caused skin irritation in animals, which is the maximum power that could be safely be used in vivo. (M. Zhou et al., J. Am. Chem. Soc., 2010, 132:15351-15358; S. Ramadan et al., Small, 2012, 8:3143-3150; B. P. Timko, et al., Adv. Materials, 2010, 22:4925-4943) Without MPPC in the bilayer, even though the temperature increases the same for liposomes containing CuS, no dye release is observed. Comparable release may be achieved at lower laser power if the drug delivery carrier were irradiated with longer wavelength light, which would improve the photothermal conversion efficiency of the CuS NP. At 800 nm, the absorption of CuS is approximately 40% of the absorption at 900 nm (FIG. 11).

### Conclusions

We present an inside-outside self-assembly process that only requires sequential mixing and simple washing and centrifugation steps to create thermosensitive, sterically stable liposome carriers with rapid contents release triggered by physiologically friendly near infra-red (NIR) light. Ethanol-induced interdigitation of DPPC (or mixed DPPC and DPPG) bilayers is used to encapsulate copper sulfide nanoparticles. The metastable phase progression used first takes advantage of the greatly increased membrane stiffness in the interdigitated phase of DPPC with added ethanol. Heating the interdigitated DPPC bilayers with CuS nanoparticles in suspension induces a phase change that softens the interdigitated bilayers, causing them to revert to closed bilayer liposomes, and in the process, capture CuS nanoparticles in the liposome interior. This co-localizes the CuS and the liposomes, so that the local heating induced by the NIR light can raise the liposome membrane temperature. The DPPC membrane is modified to include 8 to 10 mol % MPPC lysolipid and 3 to 5 mol % DSPE-PEG²⁰⁰⁰ by incubating these micelle-forming lipids with the liposomes to create a permeability transition in the membrane at ˜40° C., as well as sterically stabilize the liposomes against flocculation or opsonization in biological environments. Irradiating the CuS-lysolipid-DSPE-PEG²⁰⁰⁰-DPPC liposomes with NIR laser light power at levels well below that known to damage skin causes complete contents release from the liposomes within a few minutes. Without irradiation, contents are held for days. Previous work has shown that rapid drug release plus slight hyperthermia provides synergistic cell killing (M. Johnsson et al., Biophys. J., 2003, 85:3839-3847) that could soon be translated into new photo-triggered and targeted nanocarriers for drug release in the body based on NIR light-addressable liposomes. The new liposomes can be used to provide a rapid, localized concentration change with the spatial and temporal control provided by physiologically friendly NIR light.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1-10. (canceled)

11. A method comprising:
administering to a subject a composition comprising:
a liposome comprising lysolipid and a cargo composition at least partially encapsulated by the liposome; and
a reversibly heatable component coupled to the liposome; and
causing localized release of the cargo composition by heating the reversibly heatable component of a localized portion of the composition.

12. The method of claim 11 wherein heating the reversibly heatable component comprises exposing a localized portion of the subject to near infrared radiation.

13. The method of claim 11 further comprising stopping the release of the cargo composition by stopping the heating of the reversibly heatable component.
